(19) United States
(12) Patent Application Publication
(10) Pub. No.: US 2006/0063707 A1
(43) Pub. Date: Mar. 23, 2006

(54) Compositions for enhancing memory and methods therefor

(75) Inventors: Michel Baudry, Irvine, CA (US); Serge Bischoff, Robion (FR)

Correspondence Address: HAYNES AND BOONE, LLP, 901 MAIN STREET, SUITE 3100, DALLAS, TX, 75202, US

(73) Assignee: Lifelike Biomatic, Inc., Roanoke, TX (US)

(21) Appl. No.: 11/229,423

(22) Filed: Sep. 16, 2005

Related U.S. Application Data
(60) Provisional application No. 60/647,514, filed on Jan. 27, 2005.
(60) Provisional application No. 60/611,207, filed on Sep. 17, 2004.

(51) Int. Cl. A61K 38/16; A61K 31/549; A61K 31/538; A61K 31/198; A61K 31/165

(52) U.S. Cl. 514/8; 514/222.8; 514/230.5; 514/424; 514/561; 514/602; 514/509; 514/621

(57) ABSTRACT

Compositions for enhancing memory of a subject comprising a combination of a positive modulator of an AMPA receptor and a positive modulator of an NMDA receptor are provided, wherein each modulator of the combination is present at a subtherapeutic dose for effecting memory enhancement. Methods for using such compositions in the treatment of cognitive impairment associated with aging, age-related diseases, and CNS disorders, for example, are provided. New fusion molecules are also provided that combine the positive modulator functionalities into one molecule.

This application claims the benefit of U.S. Provisional Patent Applications No. 60/611, 207, filed Sep. 17, 2004, and No. 60/647, 514 filed Jan. 27, 2005, which applications are incorporated by reference in their entirety.

### FIELD OF THE INVENTION

The present embodiments relate to compositions and methods for treatment of cognitive impairment associated with aging, age-related diseases, and CNS disorders, for example.

### BACKGROUND

Numerous diseases associated with mild to severe impairment of learning and memory include mild cognitive impairment (MCI) associated with aging and dementias such as Alzheimer's disease. The prevalence of Alzheimer's disease and other dementias doubles every five years beyond the age of 65 (1997 Progress Report on Alzheimer's disease, National Institute on Aging/National Institute of Health). Alzheimer's disease now affects 12 million people around the world, and it is projected to increase to 22 million by 2025 and to 45 million by 2050 (Alzheimer's Association Press Release, Jul. 18, 2000).

Impairment of learning and memory is a manifestation of psychiatric disorders such as schizophrenia, attention deficit hyperactivity disorder (ADHD), and neurodegenerative diseases, such as Parkinson's disease. Currently there are no cures for these disorders and diseases and only a few drugs are commercially available to improve cognitive function.

Information storage in the brain underlies learning and memory. One of the cellular mechanisms for information storage is the phenomenon of long-term potentiation (LTP) of synaptic transmission at glutamatergic synapses. The mechanisms underlying LTP formation involve the main types of ionotropic glutamatergic receptors, the AMPA and the NMDA receptors (FIG. 1 from Lynch, G., 2002, Nature Neurosci. 5:1035-1038). LTP is elicited by delivering brief pulses of high frequency stimulation to monosynaptic connections in several defined anatomical pathways in the hippocampus and other brain structures. High frequency stimulation activates AMPA receptors and produces sufficient postsynaptic depolarization to release the NMDA receptor channel from its voltage-dependent magnesium blockade (FIG. 1, 1), resulting in an influx of calcium and a modification of the AMPA receptors (FIG. 1, 2). Increased intracellular calcium is depicted as triggering signaling pathways that modify the properties of cell adhesion molecules, thereby providing for structural modifications of synaptic contacts (FIG. 1, 3A). Signaling pathways are depicted as modifying the activity of transcription factors such as the calcium/calmodulin response element binding (CREB) protein, resulting in transcriptional responses leading to long-term modifications of cell function (FIG. 1, 3B). Activation of both AMPA and NMDA receptors is therefore key in LTP formation and, thereby, in memory formation.

A category of drugs, termed “ampakines” acts as positive modulators of AMPA receptors (PARMs). Ampakines have been proposed as cognitive enhancers due to their ability to facilitate LTP induction and to facilitate learning in a variety of tasks in mammals and humans. Ampakines are in clinical trials to treat various indications including mild cognitive impairment (MCI) associated with aging.

The NMDA receptors exhibit a variety of modulatory sites and, in particular, exhibit a binding site for the amino acid glycine. Several compounds acting at the glycine site of the NMDA receptor have also been shown to facilitate LTP formation and have been proposed as cognitive enhancers such as D-serine and D-cycloserine, for example. Further, inhibitors of glycine uptake exert similar effects as glycine, facilitate LTP formation, and are proposed as cognitive enhancers. Drugs acting as positive modulators of NMDA receptors are termed “nemdakines.”

The present embodiments address the need in the art for compositions and therapies to improve the condition of patients with cognitive impairment by improving long-term potentiation of synaptic transmission.

### SUMMARY

The present embodiments provide an unexpected synergy in the enhancement of memory upon simultaneous facilitation of AMPA and NMDA receptors. Further, the simultaneous positive modulation of the receptors provides LTP facilitation under conditions where the modulators have little or no effect by themselves.

A method for enhancing memory of a subject comprises administering to the subject a therapeutically effective amount of a combination of a positive modulator of AMPA receptors and a positive modulator of NMDA receptors, wherein each modulator of the combination is present at a subtherapeutic dose for effecting memory enhancement.

A further embodiment is a composition comprising a combination of a positive modulator of AMPA receptors and a positive modulator of NMDA receptors in a therapeutically effective amount for effecting memory enhancement, and a pharmaceutically acceptable carrier, wherein each modulator of the combination is present at a subtherapeutic dose for effecting memory enhancement.

Another embodiment is a kit comprising a container containing a composition as set forth herein and instructions for using the composition for enhancing memory in a subject.

Further compositions as embodiments of the present invention comprise a fusion molecule having positive modulating activity for both AMPA receptors and NMDA receptors, the fusion molecule comprising an ampakine functional moiety fused to a nemdakine functional moiety. A further embodiment of the present invention is a composition comprising the fusion molecule and a pharmaceutically acceptable carrier. In one embodiment, the ampakine functional moiety may be fused to a nemdakine functional moiety via a linking group, such as an alkyl group of 1-5 carbons.

A method for enhancing memory of a subject using such a fusion molecule is a further embodiment of the present invention. The method comprises administering to the subject a therapeutically effective amount of a fusion molecule having positive modulating activity for both AMPA receptors and NMDA receptors, the fusion molecule comprising an ampakine functional moiety fused to a nemdakine functional moiety; and a pharmaceutically acceptable carrier.

The ampakine functional moiety of a fusion molecule is derived from an ampakine such as azepine, a benzamide, benzoylpiperidine, benzoylpyrrolidine, benzoxazine, benzothiadiazide, benzothiadiazine, biarylpropylsulfonamide, pyrrolidinone, pyrroline, tetrahydropyridine, phenoxyacetamide, sulfur-containing organic nitrate ester, or a lectin. In one embodiment, the ampakine functional moiety is derived from the benzoylpiperidine, CX546, and in a further embodiment of a fusion molecule, the ampakine functional moiety is derived from the biarylpropylsulfonamide derivative, LY404187-NH₂.

The nemdakine functional moiety of a fusion molecule is derived from a nemdakine such as L-alanine, D-alanine, D-cycloserine, N-methylglycine, L-serine, D-serine, N, N, N-trimethylglycine, 3-amino-1-hydroxypyrrolid-2-one (HA966), (R)-(N-[3-(4′-fluorophenyl)-3-(4′-phenylphenoxy)propyl]) sarcosine (ALX5407), N-methyl-N-[3-[(4-trifluoromethyl)phenoxy]-3-phenyl-propyl]glycine (ORG 24598), a polyamine, or a neurosteroid. In one embodiment of a fusion molecule, the nemdakine functional moiety is derived from D-serine or L-serine.

Examples of fusion molecules provided herein include:
LB-217-1c having an IUPAC name of (R)-2-amino-3-[1-(2, 3-dihydro-benzo[1, 4]dioxine-6-carbonyl)-piperidin-4-yl]-2-hydroxymethyl-propionic acid,
LB-253-4c having an IUPAC name of 2-amino-2-hydroxymethyl-6-{4′-[1-methyl-2-(propane-2-sulfonylamino)-ethyl]-biphenyl-4-ylamino}-hexanoic acid, and
LB-302 having an IUPAC name of 2-amino-3-(1-benzyl-1, 2, 3, 6-tetrahydro-pyridin-4-yl)-2-hydroxymethyl-propionic acid.

Further embodiments of the invention include the use, in the preparation of a medicament for enhancing memory of a subject, of a combination of a positive modulator of AMPA receptors and a positive modulator of NMDA receptors, wherein each modulator of the combination is present at a subtherapeutic dose for effecting memory enhancement; and the use, in the preparation of a medicament for enhancing memory of a subject, of a fusion molecule having positive modulating activity for both AMPA receptors and NMDA receptors, the fusion molecule comprising an ampakine functional moiety fused to a nemdakine functional moiety.

### BRIEF DESCRIPTION OF THE DRAWINGS

FIG. 1 provides a diagram showing sites of action of modulators of AMPA receptors and NMDA receptors. FIG. 1 is from Lynch, G., 2002, Nature Neurosci. 5:1035-1038.

FIG. 2 provides a plot that demonstrates the effect of combining subthreshold concentrations of CX546 and D, L-serine on LTP in adult mice hippocampal slices. The combination produces a large facilitation of LTP under conditions where each separately does not produce facilitation of LTP. ♦, Control (means±s.e.m. of 15 slices); ∘, Combination of 20 μM CX546 and 2 μM D, L-serine (means±s.e.m. of 6 slices).

FIG. 3 provides a plot that demonstrates the effect of combining subthreshold concentrations of piracetam and D, L-serine on LTP in adult mice hippocampal slices. ●, Control (means±s.e.m. for 8 slices); ▪, Combination of 250 μM piracetam and 2 μM D, L-serine (means±s.e.m. for 6 slices). The combination produces a large facilitation of LTP under conditions where each separately does not produce facilitation of LTP.

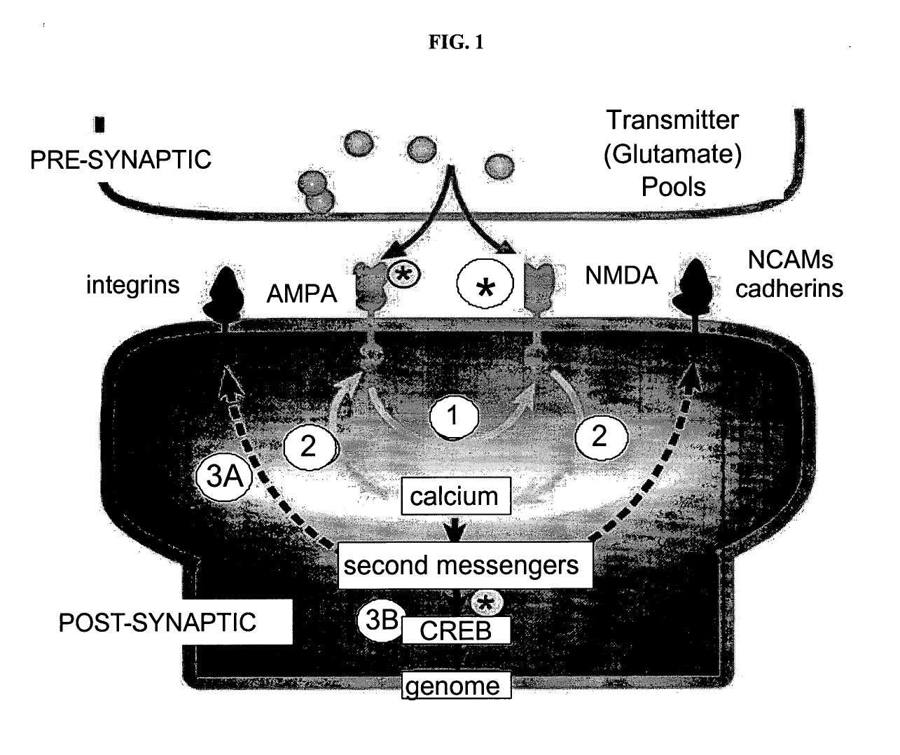

FIG. 4 provides a plot that demonstrates the in vivo effect of a combination composition of the present invention on scopolamine-induced learning deficits in rats as measured using a Morris water maze. (**p<0.001; *p<0.05; ANOVA with repeated measures followed by Bonferroni-Dunn test) The test combination drug treatment protocol was as follows:
control;
scopolamine alone (0.5 mg/kg i.p.) at t=−30 min;
piracetam alone (designated Compound A, 500 mg/kg p.o.) at t=−60 min, followed by scopolamine (0.5 mg/kg i.p.) at t=−30 min;
D-serine alone (designated Compound B, 1000 mg/kg p.o.) at t=−60 min, followed by scopolamine (0.5 mg/kg i.p.) at t=−30 min; and
a combination of piracetam, 150 mg/kg, and D-serine, 300 mg/kg, (p.o., the combination designated as LB102) at t=−60 min, followed by scopolamine (0.5 mg/kg i.p.) at t=−30 min. Data are expressed as percentage of the escape latency measured at the first trial; T1-T4 represent the 4 test trials. The combination LB-102 shows a statistically significant reversal of the scopolamine-induced deficit at the 4^{th} trial.

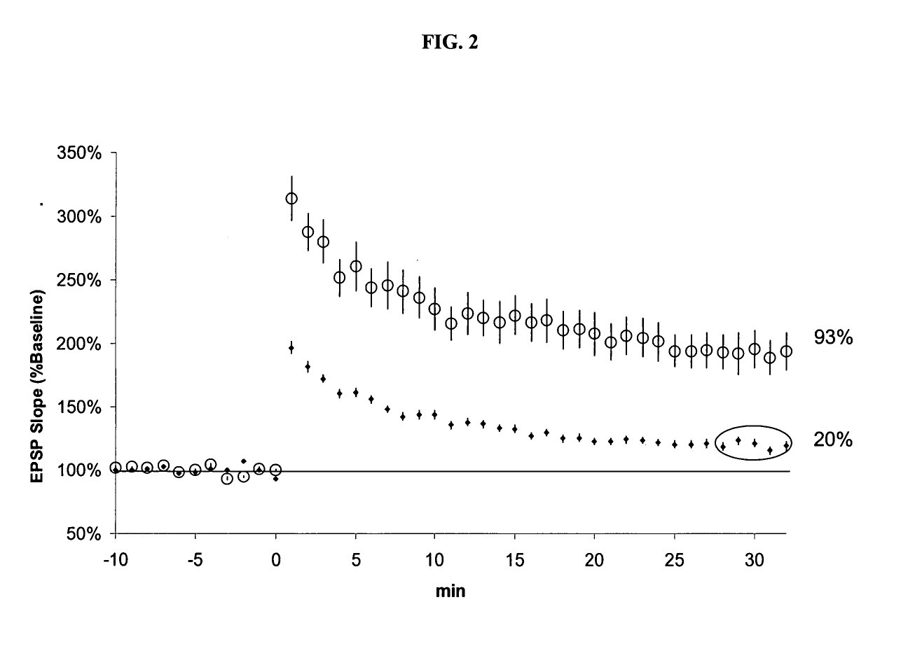

FIG. 5 provides a plot that demonstrates the effect of LB-302, a fusion molecule comprising a derivative of CX546, D-serine and an alkyl bridge of 1 carbon, on LTP. A dramatic facilitation of LTP was observed on hippocampal slices in the presence of 100 μM LB-302 (darkened circles) versus control slices without drug (lighter circles). The extent of the effect is similar to that obtained with the combination CX546 plus D, L-serine shown by FIG. 2.

### DESCRIPTION

The present embodiments provide a combination of a positive modulator of an AMPA receptor and a positive modulator of an NMDA receptor for facilitating LTP formation and, therefore, for enhancing memory. A synergy in reaching a calcium threshold required to elicit LTP is provided by such a combination at concentrations of modulators that, separately, do not trigger LTP. Further present embodiments include single molecules that combine the functionalities of a positive modulator of an AMPA receptor and a positive modulator of an NMDA receptor into one molecule for facilitating LTP formation and, therefore, for enhancing memory.

Long term potentiation is generally considered a stable increase in the strength of synaptic contacts that follows repetitive physiological activity of a type known to occur in the brain during learning. In vitro, LTP is defined by the EPSP size of single responses after brief periods of high frequency stimulation. A stable increase is generally an increase in synaptic responses lasting 30 min.

Compositions and methods of the present embodiments are useful for a condition where memory enhancement is desired such as where the subject is in need of improvement in performance of a cognitive task, for treatment of conditions associated with learning and memory impairment such as Alzheimer's, mild cognitive impairment (MCI), autism, depression, learning disorders, head injury, attention deficit hyperactivity disorder, Parkinson's and schizophrenia, for example.

Memory enhancement as a result of compositions and methods of the present embodiments for subjects having benign forgetfulness is evaluated using standard assessment instruments such as the Wechsler Memory Scale (Russell, 1975, J. Consult Clin. Psychol. 43:800-809).

Methods for diagnosing Alzheimer's Disease are provided by the National Institute of Neurological and Communicative Disorders and Stroke-Alzheimer's Disease and the Alzheimer's Disease and Related Disorders Association (NINCDS-ADRDA). The subject's cognitive function, and improvements thereof, using compositions and methods of the present embodiments is assessed by the Alzheimer's Disease Assessment Scale-cognitive subscale (ADAS-cog; Rosen et al., 1984, Am. J. Psychiatry 141:1356-1364).

Subjects are diagnosed as having autism, depression, a head injury, attention deficit hyperactivity disorder, or a learning disorder by using DSM-IV criteria APA, 1994, Diagnostic and Statistical Manual of Mental Disorders (Fourth Edition), Washington, D.C.). Improvements in such conditions as a result of treatment using compositions and methods of the present embodiments are also measured using the DSM criteria.

Parkinson's disease patients are evaluated according to the criteria described in Calne et al. (1992. Ann. Neurol. 32, pp. S125-127) and their cognitive impairment is assessed by the Mini-Mental State Examination (MMSE) (Folstein, M. F. et al., 1975. J. Psychiatr. Res. 2, pp. 189-198). Improvements in Parkinson's symptoms and cognitive impairment of patients as a result of treatment using compositions and methods of the present embodiments are also measured using the Calne and MMSE criteria.

Subjects are diagnosed as schizophrenic according to the DSM-IV criteria (APA, 1994, Diagnostic and Statistical Manual of Mental Disorders (Fourth Edition), Washington, D.C.). An evaluation of memory enhancement in a subject having schizophrenic symptoms and having been treated using compositions and methods of the present embodiments can be assessed using the Scales for the Assessment of Negative Symptoms (SANS) or Positive and Negative Syndrome Scale (PANSS) (Andreasen, 1983, Scales for the Assessment of Negative Symptoms (SANS), Iowa City, Iowa; Kay et al., 1987, Schizophrenia Bulletin 13:261-276).

A variety of accepted tests are used to determine whether a given agent is a positive modulator of an AMPA or an NMDA receptor. The primary in vitro assay is measurement of the enlargement of the excitatory postsynaptic potential (EPSP) in in vitro brain slices, such as rat hippocampus brain slices. Modulators useful in the present embodiments are agents that cause an increased ion flux through the AMPA or NMDA receptor complex channels. Increased ion flux is typically measured as at least a 10% increase in decay time, amplitude of the waveform and/or the area under the curve of the waveform and/or a decrease of at least 10% in rise time of the waveform, for example.

A schematic representation for activity of an ampakine receptor or activity for a nemdakine receptor is indicated below.
agonist A+receptor R [symbol] closed complex AR [symbol] open complex AR* [symbol] densensitized ARd

In this model agonist A of the receptor interacts with receptor R and forms a complex, AR. The binding induces opening of the channel AR* followed by a transition to a desensitized state ARd. All of the reactions are reversible. A positive modulator of an NMDA receptor or a positive modulator of an AMPA receptor may modify this kinetic scheme in a number of ways including:
i) accelerating the rate of channel opening, that is, the transition AR to AR*,
ii) slowing the rate of closing of the channel, that is, the transition AR* to AR,
iii) blocking desensitization, that is, preventing the transition AR* to Ard,
iv) slowing the rate of desensitization, that is the transition AR* to Ard, or
v) accelerating the rate of recovery from desensitization, that is the transition ARd to R, or to AR*, or to AR.

In each of cases i)-v), the positive modulator of the receptor increases the amount of ions that are able to move through the channel.

The positive modulators of the AMPA receptor or the NMDA receptor of the present embodiments may have one or more of the above cited mechanisms of action or a mechanism that is yet to be elucidated. Known positive modulators of AMPA receptors and known positive modulators of NMDA receptors include agents as follows.

Positive Modulators of AMPA Receptors: For a general review of AMPA receptor modulators, see Yamada (1998, Neurobiology of Disease 5:67-80).

Positive modulators of AMPA receptors include, for example, an azepine, a benzamide, benzoylpiperidine, benzoylpyrrolidine, benzoxazine, benzothiadiazide, benzothiadiazine, biarylpropylsulfonamide, pyrrolidinone, pyrroline, tetrahydropyridine, phenoxyacetamide, sulfur-containing organic nitrate ester, lectin, a salt thereof, an ester thereof, a precursor thereof, a metabolite thereof, a derivative thereof, a racemic mixture thereof, or a combination thereof.

Examples of an azepine include an (R)-7-fluoro-2, 3, 11, 11a-tetrahydro-1H, 5H-pyrrolo[2, 1-c][1, 4]benzoxazepine-5-one, (S)-7-fluoro-2, 3, 11, 11a-tetrahydro-1H, 5H-pyrrolo[2, 1-c][1, 4]benzox-azepine-5-one, (S)-9-fluoro-2, 3, 11, 11a-tetrahydro-1H, 5H-pyrrolo[2, 1-c][1, 4]-benzoxazepine-5-one, (R)-9-fluoro-2, 3, 11, 11a-tetrahydro-1H, 5H-pyrrolo[2, 1-c][1, 4]benzoxazepine-5-one, and (S)-6-fluoro-2, 3, 11, 11a-tetrahydro-1H, 5H-pyrrolo[2, 1-c][1, 4]benzoxazepine-5-one, a salt thereof, an ester thereof, a precursor thereof, a metabolite thereof, a derivative thereof, a racemic mixture thereof, or a combination thereof, for example.

Examples of a benzoylpiperidine include a 1-(quinoxalin-6-ylcarbonyl)piperidine (CX516, BDP-12) (Arai et al., 2002, J. Pharmacol Exp. Ther 303:1075-1085; Arai et al., 2004, Neuroscience 123:1011-1024; Nagarajan et al., 2001, Neuropharmacol. 41:650-663), 1-(1, 4-benzodioxan-6-ylcarbonyl)piperidine (CX546) (Arai et al., 2002, 2004 ibid; Nagarajan et al., 2001 ibid), 1-(4′-methoxymethylbenzoyl)piperidine, 1-(3′-methoxymethylbenzoyl)piperidine, 1-(4′-ethoxymethylbenzoyl)piperidine, 1-(4′-hydroxymethylbenzoyl)piperidine, 1-(4′-(3″, 4″-methylenedioxyphenoxy)-methylbenzoyl)piperidine, 1-(1, 3-benzodioxol-5-ylcarbonyl)-piperidine (1-BCP) (Stäubli et al., 1994, PNAS 91:11158-11162), (R, S)-1-(2-methyl-1, 3-benzodioxol-5-ylcarbonyl)-piperidine, 1-(1, 3-benzoxazol-6-ylcarbonyl)-piperidine, 1-(1, 3-benzimidazol-5-ylcarbonyl)-piperidine, benzoylpiperidine of U.S. Pat. No. 5, 891, 876, Apr. 6, 1999 to Lynch et al. and of U.S. Pat. No. 5, 650, 409, Jul. 22, 1997 to Rogers et al. (both patents are incorporated by reference herein in their entirety), a salt thereof, an ester thereof, a precursor thereof, a metabolite thereof, a derivative thereof, a racemic mixture thereof, or a combination thereof, for example.

Examples of a benzoxazine include a 2H, 3H, 6aH-pyrrolidino[2″, 1″-3′2′]1, 3-oxazino[6′, 5′-5, 4]benzo[e]1, 4-dioxan-10-one (CX614) (Arai et al., 2000, Mol. Pharmacol. 58:802-813), (R, S)-6-methoxymethyl-2, 3-dihydro-1H-pyrrolo[2, 1-b][1, 3]benzoxazine-9(3aH)-one, R, S)-7-methoxymethyl-2, 3-dihydro-1H-pyrrolo[2, 1-b][1, 3]benzoxazine-9(3aH)-one, 7, 8-dihydro-5aH, 10H-1, 3-dioxolo[4, 5-g]oxazolo[2, 3-b][1, 3]benzoxazin-10-one (1), 8, 9-dihydro-6aH, 11H-1, 4-dioxan[2, 3-g]oxazolo[2, 3-b][1, 3]benzoxazin-11-one, 7, 8-dihydro-2, 2-dimethyl-5aH, 10H-1, 3-dioxolo[4, 5-g]oxazolo[2, 3-b][1, 3]benzoxazin-10-one, 7, 8-dihydro-5aH, 10H-1, 3-dioxolo[4, 5-g]thiazolo[2, 3-b][1, 3]benzoxazin-10-one, 7, 8-dihydro-7-methyl-5aH, 10H-1, 3-dioxolo[4, 5-g]oxazolo[2, 3-b][1, 3]benzoxazin-10-one, 7, 8-dihydro-8-methyl-5aH, 10H-1, 3-dioxolo[4, 5-g]oxazolo[2, 3-b][1, 3]benzoxazin-10-one, 8, 9-dihydro-5aH, 7H, 10H-1, 3-dioxolo[4, 5-g][1, 3]oxazino[2, 3-b][1, 3]benzoxazin-11-one, 7, 8-dihydro-5a-methyl-10H-1, 3-dioxolo[4, 5-g]oxazolo[2, 3-b][1, 3]benzoxazin-10-one, benzoxazine of U.S. Pat. No. 5, 985, 871, Nov. 16, 1999 to Rogers et al. (incorporated by reference herein in its entirety), a salt thereof, an ester thereof, a precursor thereof, a metabolite thereof, a derivative thereof, a racemic mixture thereof, or a combination thereof, for example.

Examples of a benzoylpyrrolidine include 2H, 5aH-pyrrolidino[2″, 1″-3′, 2′]1, 3-oxazino[6′, 5′-5, 4]benzo[d]1, 3-dioxolan-9-one (BDP-20, CX554) (Arai et al., 1996, Neuroscience 75:573-585), 1(1, 3-benzodioxol-5-ylcarbonyl)-pyrrolidine), benzoylpyrrolidine of U.S. Pat. No. 5, 650, 409, Jul. 22, 1997 to Rogers et al. (incorporated by reference herein in its entirety), a salt thereof, an ester thereof, a precursor thereof, a metabolite thereof, a derivative thereof, a racemic mixture thereof, or a combination thereof, for example.

Examples of a biarylpropylsulfonamide include N-2-(4-(3-thienyl)phenyl)propyl-2-propanesulfonamide (LY392098) (Gates et al., 2001, Neuropharmacol. 40:984-991), N-2-(4-(cyanophenyl)phenyl)propyl-2-propanesulfonamide (LY404187) and a 22h derivative thereof (Ornstein et al., J. Med. Chem. 2000, 43, 4354-4358; Gates et al., ibid), (R)-4′-[1-fluoro-1-methyl-2-(propane-2-sulfonylamino)-ethyl]-biphenyl-4-carboxylic acid methylamide (LY503430) (O'Neill et al., 2004, Curr. Med. Chem. 4 :95-103), a salt thereof, an ester thereof, a precursor thereof, a metabolite thereof, a derivative thereof, a racemic mixture thereof, or a combination thereof.

Examples of a benzothiadiazide include a cyclothiazide (Partin et al., 1996, J. Neurosci. 16:6634-6647; Patneau et al., 1993, J. Neurosci. 13:3496-3509), diazoxide (Vyckliky et al., 1991, Neuron 7:971-984; Yamada and Rothman, 1992, J. Physiol. 458:385-407), IDRA21 (Bertolino et al., 1993, Receptors Channels. 1(4):267-78), Buccafusco et al., 2004, Neuropharmacol. 46:10-22; Phillips et al., 2002, Bioorg & Med. Chem. 10:1229-1248; Pirotte et al., 1998, J. Med. Chem. 41:2946-2951), bendroflumethiazide, benzthiazide, buthiazide, chlorothiazine, epithiazide, hydrochlorothiazide, hydroflumethiazide, methylclothiazide, methalthiazide, polythiazide, trichlormethiazide, 5-ethyl-benzothiadiazide (compound D1), a salt thereof, an ester thereof, a precursor thereof, a metabolite thereof, a derivative thereof, a racemic mixture thereof, or a combination thereof, for example.

Examples of a benzothiadiazine include 7-chloro-3-methyl-3-4-dihydro-2H-1, 2, 4 benzothiadiazine S, S, dioxide, (S)-2, 3-dihydro-[3, 4]cyclopentano-1, 2, 4-benzothiadiazine-1, 1-dioxide (S18986-1) (Desos et al., 1996, Bioorg. Med. Chem. 6:3003; Dicou et al., 2003, Brain Res. 970:221-225), a salt thereof, an ester thereof, a precursor thereof, a metabolite thereof, a derivative thereof, a racemic mixture thereof, or a combination thereof, for example.

Examples of a pyrrolidinone include aniracetam (N-anisoyl-2-pyrrolidinone) (Ito et al., 1990, J. Physiol. 424:533-543; Partin et al., 1996, J. Neurosci. 16:6634-6647; Lawrence et al., 2003, Mol Pharmacol. Aug. 64(2):269-78), piracetam (Copani et al., 1992, J. Neurochem. 58:1199-1204), oxiracetam (Copani et al., ibid), (R)-1-p-anisoyl-3-hydroxy-2-pyrrolidinone (AHP), a salt thereof, an ester thereof, a precursor thereof, a metabolite thereof, a derivative thereof, a racemic mixture thereof, or a combination thereof.

Examples of a tetrahydropyridine include 1-(1, 4-benzodioxan-5-ylcarbonyl)-1, 2, 3, 6-tetrahydropyridine, N-(4-dimethylamino)benzoyl-1, 2, 3, 6-tetrahydropyridine, 1-(1, 3-benzodioxol-5-ylcarbonyl)-1, 2, 3, 6-tetrahydropyridine, 1-(1, 3-benzoxazol-6-ylcarbonyl)-1, 2, 3, 6-tetrahydopyridine, 1-(1, 3-benzoxazol-5-ylcarbonyl)-1, 2, 3, 6-tetrahydropyridine, 1-(guinoxalin-6-ylcarbonyl)-1, 2, 3, 6-tetrahydropyridine, tetrahydropyridine of U.S. Pat. No. 5, 891, 876, Apr. 6, 1999 to Lynch et al. (incorporated by reference herein in its entirety), a salt thereof, an ester thereof, a precursor thereof, a metabolite thereof, a derivative thereof, a racemic mixture thereof, or a combination thereof, for example.

Examples of a pyrroline include 1-(1, 4-benzodioxan-5-ylcarbonyl)-3-pyrroline, pyrroline of U.S. Pat. No. 5, 891, 876, Apr. 6, 1999 to Lynch et al. (incorporated by reference herein in its entirety), a salt thereof, an ester thereof, a precursor thereof, a metabolite thereof, a derivative thereof, a racemic mixture thereof, or a combination thereof.

Examples of a lectin include concanavalin A, wheat germ agglutinin, a salt thereof, an ester thereof, a precursor thereof, a metabolite thereof, a derivative thereof, a racemic mixture thereof, or a combination thereof. Lectins appear to reduce desensitization by binding to glycosylation sites of AMPA receptors (Everts et al., Mol. Pharmacol. 52:861-873; Viklicky et al., ibid).

Examples of a phenoxyacetamide include 4-[2-(phenylsulfonylamino)ethylthio]-2, 6-difluoro-phenoxyacetamide (PEPA) (Sekiguchi et al., 1997, J. Neurosci 17:5760-5771; Sekiguchi et al., 2002, Br. J. Pharmacol. 1361:033-1041), a salt thereof, an ester thereof, a precursor thereof, a metabolite thereof, a derivative thereof, a racemic mixture thereof, or a combination thereof.

Examples of a sulfur-containing organic nitrate ester include GT-21-005 (Lei et al., 2001, J. Neurophysiol. 85:2030-2038), a salt thereof, an ester thereof, a precursor thereof, a metabolite thereof, a derivative thereof, a racemic mixture thereof, or a combination thereof. Sulfur-containing organic nitrate esters appear to reduce the sensitivity of AMPA receptor desensitization.

A positive modulator of an AMPA receptor likely acts on a transition state of the AMPA receptor complex by reducing deactivation, slowing channel closing, accelerating channel opening, reducing/blocking desensitization or accelerating the recovery from desensitization, for example. Modulation may occur at or near the dimer interface, at levels downstream of the receptor channel involving proteins linked to the postsynaptic densities (PSD) and to proteins engaged in the cascade of second messengers and even further downstream to transcriptional and translational mechanisms involving, among others, CREB (Lynch, 2002, Nature Neurosci. 5:1035-1038).

A positive modulator of an AMPA receptor may also be an agent having activity for reducing an effect of a negative modulator. For example, an agent having activity for soaking up protons is a positive modulator since protons promote receptor desensitization (Lei et al., ibid). An agent that deactivates thiocyanate is also a positive modulator of AMPA receptors (Arai et al., 1995, Neuroscience 66:815-827; Partin et al., ibid). A positive modulator of an AMPA receptor may also be an agent that reduces the effect of a noncompetitive antagonist (also called negative allosteric modulators, Barreca et al., 2003, J. Chem. Inf. Comput. Sci. 43:651-655) and derivatives thereof such as 1-4-aminophenyl-methyl-7, 8-methylenedioxy-5H-2, 3-benzodiazepine (GYKI 52466) (Vizi et al., 1996, CNS Drug Rev. 2:91-126), quinoxaline-7sulphonamide, NS102, 5-nitro-6, 7, 8, 9-tetrahydrobenzo[g]-2, 3-dione-3-oxime (SYM-2206), (7-acetyl-5-(4-aminophenyl)-8-methyl-8, 9-dihydro-7H-1, 3-dioxolo[4, 5-b][2, 3]benzodiazepine (Talampanel), 3-(2-chloro-phenyl)-2-[2-(6-diethylaminomethyl-pyridin-2-yl)-vinyl]-6-fluoro-3H-quinazolin-4-one (CP-465022), or chemical analogs using the catalyst approach as cited by Barreca et al. (2003, J. Chem. Inf. Comput. Sci. 43:651-655).

A Positive Modulator of an NMDA receptor: A positive modulator of an NMDA receptor may affect any of a number of interactions among the NMDA receptor, glycine and glutamate as shown in the following scheme according to Lester et al. (1993, J. Neurosci. 13:1088-1096).

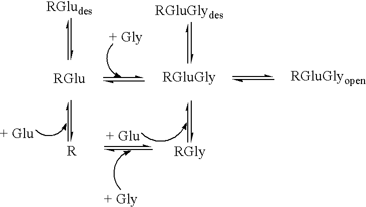

In this scheme, “R” is the NMDA receptor, “Gly” is glycine, “Glu” is glutamate, “des” depicts a desensitized state of a receptor complex, and open depicts a receptor having a channel open for calcium ions to pass.

Positive modulators of an NMDA receptor include L-alanine, D-alanine, D-cycloserine, N-methylglycine, L-serine, D-serine, N, N, N-trimethylglycine, 3-amino-1-hydroxypyrrolid-2-one (HA966), (R)-(N-[3-(4′-fluorophenyl)-3-(4′-phenylphenoxy)propyl])sarcosine (ALX5407), N-methyl-N-[3-[(4-trifluoromethyl)phenoxy]-3-phenyl-propyl]glycine (ORG 24598), a polyamine, neurosteroid, a salt thereof, an ester thereof, a precursor thereof, a metabolite thereof, a derivative thereof, a racemic mixture thereof, or a combination thereof, for example.

Such positive modulators may have a mechanism of action as follows.

Agonist at the glycine site: Positive modulators at the glycine site are likely located on the NR1 subunit of the NMDA receptor. Glycine acts as a co-agonist with glutamate; neither glutamate nor glycine alone can activate the NMDA receptor. While glutamate increases the rate of dissociation of glycine from the NMDA receptor, the partial agonist at the glycine site HA966 reduces the affinity of glutamate for the NMDA receptor also by increasing its dissociation rate. Since binding of glutamate and glycine are necessary for channel opening and thus, for synaptic activation, the influence of one by the other has necessarily an impact on the transition states of the kinetic scheme (Lester et al., ibid.). Positive modulators of the glycine site include D-serine, L-alanine, L-serine, 3-amino-1-hydroxypyrrolid-2-one (HA966), D-cycloserine, and derivatives thereof.

Blockers of glycine uptake/transport: Positive modulators of the glycine transporter site that block the re-uptake/transport of glycine out of the synaptic cleft, thereby increasing concentrations of glycine include (R)-(N-[3-(4′-fluorophenyl)-3-(4′-phenylphenoxy)propyl])sarcosine (ALX5407) (Kemp and McKeman, 2002, Nature Neurosci. 5:1039-1042), N-methyl-N-[3-[(4-trifluoromethyl)phenoxy]-3-phenyl-propyl]glycine (ORG 24598) (Kemp and McKeman, ibid.), and derivatives thereof.

Compounds acting at other modulatory sites of the NMDA receptor complex: Positive modulators of channel sites include agents that reduce activity for Mg2+, for PCP, for MK801, and for ketamine, for example. Positive modulators of sites on the NR2 subunits include agents that reduce activity for Zn2+, and for protons (Jang et al., 2004, PNAS 101:8198-8203). Positive modulators at the NR2 subunits include polyamines such as spermine, spermidine, neomycine, for example, that enhance synaptic activity by preventing the proton-induced inhibition of receptor activity; neurosteroids, in particular, pregnenolone sulphate acts on a segment of the extracellular domain next to a transmembrane portion called SMD1 (steroid modulatory domain 1) (Jang et al., ibid.); ATP (Kloda et al., 2004, Mol. Pharmacol. 65:1386-1396), and derivatives thereof. Further positive modulators of the NR2 subunits include agents for preventing Ca2+ dependent calmodulin-sensitive and calmodulin-insensitive inactivation of NMDA receptor activity (Rycroft and Gibb, 2002, J. Neurosci. 22:8860-8868; Vissel et al., 2002, Mol. Pharmacol. 61:595-605). Additional positive modulators of the NR2 subunits signal intracellular proteins that convey to the second messenger cascade via the proteins anchored to the postsynaptic density (PSD95) and other mechanisms downstream leading to receptor trafficking (Kemp and McKeman, ibid.).

For positive modulators of either receptor, terms as used herein are defined as follows.

The term “a salt thereof” means a salt such as a sodium salt, a potassium salt, a calcium salt, a magnesium salt, a zinc salt, or an ammonium salt of the modulator, for example.

The term “an ester thereof” means having an ester linkage to a C1-C20 carbon group, for example.

The term “a derivative thereof” means having a substituent bonded to the ampakine or nemdakine such as an alkyl, alkenyl, alkynyl, aryl, alkylaryl, formyl, halo, acyl, hydroxyalkyl, hydroxyalkoxy, hydroxyalkenyl, hydroxyalkynyl, saccharide, carboxy, carboxyalkyl, carboxyamide, carboxyamidealkyl, alkyl sulfoxide, alkyl sulfone, alkyl sulfide, tetrahydropyran, tetrahydrothiapyran, thioalkyl, halo, haloalkyl, haloalkenyl, haloalkynyl, alkyl ester, aminoalkyl, phosphoalkyl, N-oxide, dialkylamino, carbamate, or arylsulfonyl, for example. An alkyl, alkenyl, or alkynyl group may have up to about 20 carbons.

As used herein, “a therapeutically effective amount” means the concentration or quantity or level of the nemdakine and ampakine in combination that can affect LTP in a patient in need thereof. With regard to use of fusion molecules as used herein, “a therapeutically effective amount” means the concentration or quantity or level of the fusion molecule that can affect LTP in a patient in need thereof. The specific “therapeutically effective amount” will vary with such factors as the particular condition being treated, the physical condition of the patient, the duration of the treatment, the nature of concurrent therapy (if any), the specific formulations employed and the form of the agents.

As used herein, the term “precursor” refers to a form or derivative of a positive modulator that has minimal therapeutic activity until it is converted to its desired biologically active form. A precursor is a compound having one or more functional groups or carriers covalently bound thereto, which functional groups or carriers are removed from the compound by metabolic processes within the body to form the respective bioactive compound. Examples of a precursor include a phosphorylated derivative or a methylated derivative of a modulator. An example of a precursor of D-serine is D-phosphoserine or L-phosphoserine, for example, and an example of a precursor of glycine is N, N, N-trimethylglycine (betaine), or N, N-dimethylglycine.

As used herein, the term “metabolite” refers to the break-down or end product of a positive modulator produced by biotransformation in the patient body, e.g., biotransformation to a more polar molecule such as by oxidation, reduction, or hydrolysis, or to a conjugate (see Goodman and Gilman, “The Pharmacological Basis of Therapeutics” 8.sup.th Ed., Pergamon Press, Gilman et al. (eds.), 1990 for a discussion of biotransformation). As used herein, the metabolite of a modulator may be the biologically active form of the compound in the body. An assay for activity of a metabolite of a modulator of the present embodiments is known to one of ordinary skill in the art, for example, testing for long term potentiation, or for cognitive improvement.

As used herein, a “subject” is a patient in need of memory enhancement. The subject may have symptoms of memory impairment, or may have symptoms of a neurodegenerative disease. The subject may have symptoms of cognitive impairment due to aging, Alzheimer's disease, dementia, schizophrenia, attention deficit hyperactivity disorder, or Parkinson's disease. The subject may be in need of improvement in performance of a cognitive task. Subjects include humans, domesticated animals, or laboratory animals, for example.

Fusion Molecule: A “fusion molecule, ” as provided herein, is a molecule having positive modulating activity for both AMPA receptors and NMDA receptors. A fusion molecule comprises an ampakine functional moiety fused to a nemdakine functional moiety. The ampakine functional moiety and the nemdakine functional moiety are optionally separated by a linker region.

An ampakine functional moiety is derived from, for example, an azepine, a benzamide, benzoylpiperidine, benzoylpyrrolidine, benzoxazine, benzothiadiazide, benzothiadiazine, biarylpropylsulfonamide, pyrrolidinone, pyrroline, tetrahydropyridine, phenoxyacetamide, sulfur-containing organic nitrate ester, or a lectin. An ampakine functional moiety may be derivatized with groups as defined supra.

A nemdakine functional moiety is derived from L-alanine, D-alanine, D-cycloserine, N-methylglycine, L-serine, D-serine, N, N, N-trimethylglycine, 3-amino-1-hydroxypyrrolid-2-one (HA966), (R)-(N-[3-(4′-fluorophenyl)-3-(4′-phenylphenoxy)propyl])sarcosine (ALX5407), N-methyl-N-[3-[(4-trifluoromethyl)phenoxy]-3-phenyl-propyl]glycine (ORG 24598), a polyamine, or a neurosteroid, for example. A nemdakine functional moiety may be derivatized with groups as defined supra.

A linker region may be used to bond the ampakine functional moiety and the nemdakine functional moiety. A linker region may be described as a couple, i.e. a product formed by reaction of a reactive group designed to attach covalently an ampakine moiety and a nemdakine moiety. Exemplary linkers or couples are alkyl or aryl groups having amide, amine, disulfide, thioether, ether, ester, or phosphate reactive groups. The linker region is typically an alkyl group having 1, 2, 3, 4, or 5 carbons, isomers thereof, aryl groups, or alkylaryl groups where the alkyl has 1, 2, 3, 4, or 5 carbons.

As provided by Example 3 herein, the functional moiety of the benzoylpiperidine ampakine, CX546, is essentially the complete molecule of CX546. The functional moiety of the nemdakine, D-serine, is essentially the complete molecule of D-serine. The linker is a one carbon unit provided by a 4-bromomethyl piperidine t-butyl ester in a condensation reaction with a phenyl oxazoline derivative of serine benzyl ester.

For fusion molecules having a general formula A-1 or A-2 as shown below, the piperidine ring is independently substituted in the 2-, 3- or 4-position, X is an alkyl group of one to five carbon atoms and R1, R2, R3, R4, R5 and R6 are as defined below.

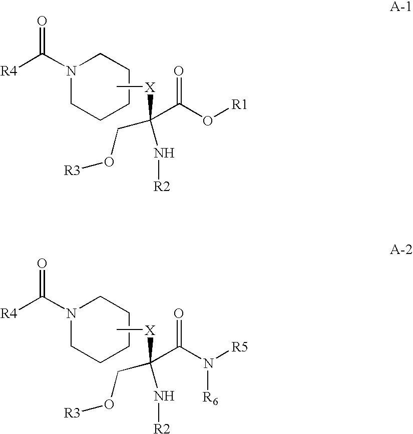

R1 is C1-C4 alkyl such as methyl or ethyl, or aryl such as benzyl. R2 and R3 are independently H, formyl or acyl thereby providing common prodrug modifications. R5 and R6 are independently H, acyl, formyl, alkyl such as methyl or ethyl, or aryl such as benzyl.

R4 is a derivative of benzoic acid (such as shown by formula IX below), or of a five- or six-membered heterocyclic carboxylic acid with one or two rings, such as a thiophene-2-carboxylic acid (formula X), thiophene-3-carboxylic acid (formula XI), pyridine-2-carboxylic acid (formula XII), pyridine-3-carboxylic acid (formula XIII) or pyridine-4-carboxylic acid (formula XIV), pyrimidine-2-carboxylic acid (formula XV), pyrimidine-4-carboxylic acid (formula XVI) or pyrimidine-5-carboxylic acid (formula XVII), pyrazine-2-carboxylic acid (formula XVIII), 2, 3-dihydro-benzo(1, 4)dioxine-6-carboxylic acid (formula XIX), 1-quinoxalin-2-carboxylic acid (formula XX) or 1-quinoxalin-6-carboxylic acid (formula XXI). R4 is optionally substituted with one or two groups R7 wherein each R7 is independently halo, such as fluoro, chloro or bromo; alkoxy such as methoxy, or ethoxy; alkyl such as methyl or ethyl; or cyano; with the provisos that R7 is other than methoxy or ethoxy in the cases of formula X and XI; and a halo is in other than an ortho position to a nitrogen atoms in the heterocycles XII-XVIII and XX-XXI.

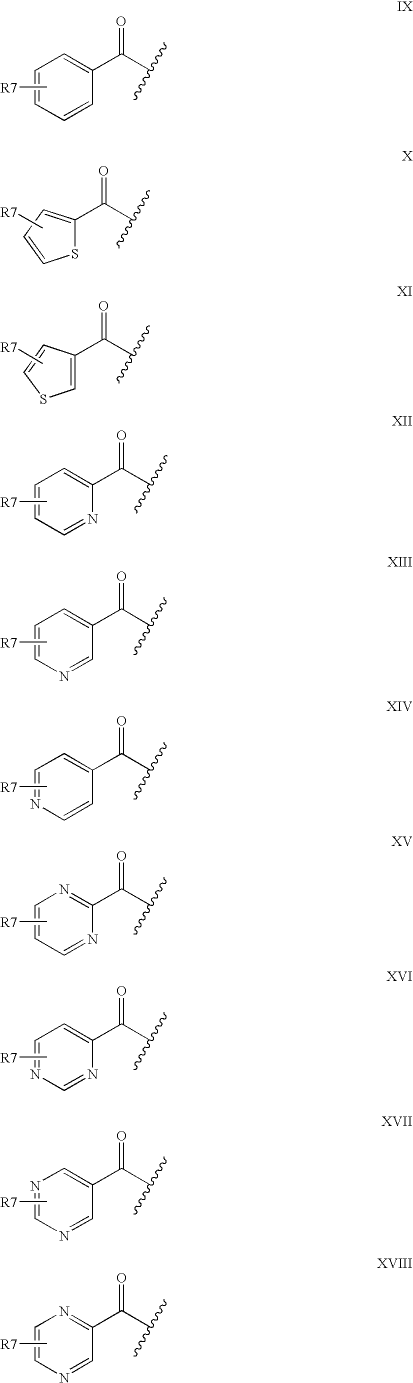

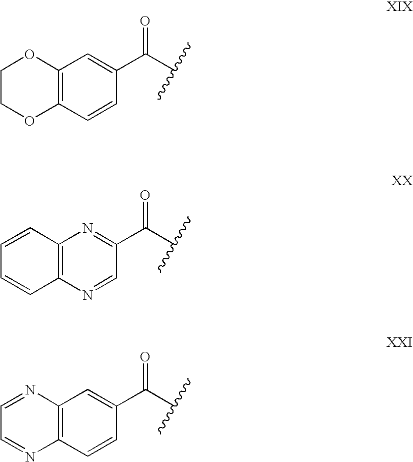

In one embodiment of a fusion molecule of formula A-1 or A-2, the amino acid substituent is at the 4-position of the piperidine ring as shown by the compounds VII and VIII.

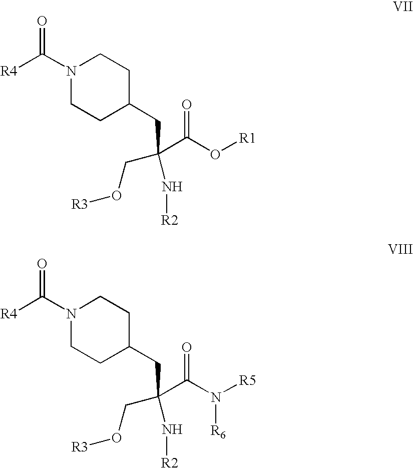

One of ordinary skill in the art of organic synthesis recognizes that a series of derivatives are produced by routine modifications of the above cited scheme. Such derivatives are also provided by the present invention as molecules having the dual functionality described herein. The amino acid part of the substance can be in the unprotected, partially protected or fully protected form. The compounds can be obtained as diastereomeric mixtures, racemates or enantiomers.

As provided by Example 4 herein, the functional moiety of the biarylpropylsulfonamide ampakine, LY404187-NH₂, is essentially the complete molecule, particularly, the isopropylsulfonamide portion, with the amino group of the biaryl portion providing a linkage to D-serine. The functional moiety of the nemdakine, D-serine, is essentially the complete molecule of D-serine. The linker is a four carbon unit provided by dibromo-n-butane in a condensation reaction.

As provided by Example 5 herein, the functional moiety of the benzoylpiperidine ampakine, CX546, is the 2-amino-3-1-benzyl-1, 2, 3, 6-tetrahydropyridine portion of the CX546 molecule. The functional moiety of the nemdakine, D-serine, is essentially the complete molecule of D-serine. The linker is a one carbon unit provided by a 4-bromomethyl piperidine t-butyl ester in a condensation reaction with a phenyl oxazoline derivative of serine benzyl ester.

Fusion of an ampakine functional moiety and a nemdakine functional moiety takes place at a position of each moiety so as to preserve the functionality of the moieties. For example, the sulfonamide portion of a biarylpropylsulfonamide is a determinant for ampakine activity. Such a determinant is preserved in a fusion scheme. The size of the linker is guided by the relative positions of the binding pockets of the ampakine and nemdakine receptor.

Dosages: Fusion molecule compositions and combination compositions of the present embodiments are administered to a subject at a therapeutically effective dosage that enhances memory. Enhancement of memory is evaluated by a number of diagnostic measures as set forth above.

The positive modulators of the present embodiments chosen for a particular patient, the carrier and the amount will vary widely depending on the patient, the type of memory impairment, the pharmacodynamic characteristics of the modulators and their mode and route of administration, the age, health, and weight of the patient, the nature and extent of symptoms, the metabolic characteristics of the combination and of the patient, the kind of concurrent treatment, the frequency of treatment, or the effect desired.

In general, the positive modulator of an NMDA receptor and the positive modulator of an AMPA receptor in the combination is each at an amount that is subtherapeutic. The term “subtherapeutic, ” as used herein, means that each modulator is itself present at a lower dose than the dosage that is typically used for treatment with the modulator alone for effecting memory enhancement i.e., a “therapeutic dose.” The amount may be less than, or an amount between any of and including any of 90%, 80%, 70%, 60%, 50%, 40%, 30%, 20%, 15%, 10%, or 5% of a therapeutic dose. In one embodiment, the subtherapeutic dose of an ampakine or of a nemdakine in the combination is a dose at one-half or less than one-half of a therapeutic dose. In a further embodiment, the subtherapeutic dose of an ampakine or of a nemdakine in the combination is a dose at one-fifth or less than one-fifth of a therapeutic dose. An appropriate dosage can be determined by one of ordinary skill in the art by monitoring the patient for signs of memory improvement for example, as cited herein, and adjusting the dosage as needed.

A subtherapeutic dosage for systemic administration of a positive modulator of an NMDA receptor ranges from about 0.1 mg to about 1 g per kg weight of subject per administration. A subtherapeutic dosage of such a positive modulator in the combination of the present embodiments is between about and including any of 0.1 mg, 0.5 mg, 1 mg, 2 mg, 5 mg, 10 mg, 15 mg, 20 mg, 25 mg, 50 mg, 100 mg, 150 mg, 200 mg, 300 mg, 400 mg, 500 mg, or 1000 mg per kg weight of subject per administration.

A subtherapeutic dosage for systemic administration of a positive modulator of an AMPA receptor range from about 0.1 mg to about 1 g per kg weight of subject per administration. A subtherapeutic dosage of such a positive modulator in the combination of the present embodiments is between about and including any of 0.1 mg, 0.5 mg, 1 mg, 2 mg, 5 mg, 10 mg, 15 mg, 20 mg, 25 mg, 50 mg, 100 mg, 150 mg, 200 mg, 300 mg, 400 mg, 500 mg, or 1000 mg per kg weight of subject per administration.

The combination of positive modulators may be administered to the subject simultaneously or sequentially as long as an overlap in persistence time exists between the administrations. The positive modulators may be combined in a single composition or as two or more individual compositions.

In general, a fusion molecule is administered at a therapeutically effective amount as defined herein. A therapeutically effective amount of a fusion molecule provides an ampakine functional moiety and a nemdakine functional moiety at a lower dose than the dosage that is typically used for treatment with an ampakine or a nemdakine alone for effecting memory enhancement as set forth above.

The dosage for humans is generally less than that used in mice for experimental studies and is typically about 1/12 of the dose that is effective in mice. Thus, if 500 mg/kg was effective in mice, a dose of 42 mg/kg would be used in humans.

A dosage unit contains from about 1 mg to about 1000 mg of the active combination or the fusion molecule. The active ingredient is generally present in an amount of about 0.5% to about 95% by weight based on the total weight of the dosage unit. Intravenously, doses may range from about 1 to about 10 mg/kg/minute during a constant rate infusion.

Formulations: Formulations of the present embodiments include a fusion molecule as set forth herein, or a combination of a positive modulator of an AMPA receptor and a positive modulator of an NMDA receptor generally mixed with a pharmaceutically acceptable carrier. A “pharmaceutically acceptable carrier” is a pharmaceutically acceptable solvent, suspending agent or vehicle for delivering the combination to the subject. The carrier may be liquid or solid and is selected with the planned manner of administration in mind. A “pharmaceutically acceptable” carrier is one that is suitable for use with humans and/or animals without undue adverse side effects commensurate with a reasonable benefit/risk ratio.

Oral formulations suitable for use in the practice of the present embodiments include capsules, time-release capsules, gels, cachets, tablets, powders, granules, solutions, suspensions, liquid emulsions, a bolus, an electuary, or a paste.

Generally, formulations are prepared by uniformly mixing the combination or the fusion molecule with liquid carriers or finely divided solid carriers or both and then, if necessary, shaping the product.

Examples of suitable solid carriers include lactose, sucrose, gelatin, agar and bulk powders, starch, glucose, methyl cellulose, magnesium stearate, dicalcium phosphate, calcium sulfate, mannitol, sorbitol, cyclodextrin, cyclodextrin derivatives, or the like.

Examples of suitable liquid carriers include water, pharmaceutically acceptable fats and oils, alcohols or other organic solvents including esters, emulsions, syrups or elixirs, suspensions, solutions, suspensions, solution or suspension reconstituted from non-effervescent granules or from effervescent granules, solution or suspension reconstituted from non-effervescent granules or from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils, or polyethylene glycols.

Capsule or tablets can be easily formulated and can be made easy to swallow or chew. Tablets may contain suitable carriers, binders, lubricants, diluents, disintegrating agents, coloring agents, flavoring agents, flow-inducing agents, or melting agents. A tablet may be made by compression or molding, optionally with one or more additional ingredients. Compressed tables may be prepared by compressing the active ingredient in a free flowing form (e.g., powder, granules) optionally mixed with a binder (e.g., gelatin, hydroxypropylmethylcellulose), lubricant, inert diluent, preservative, disintegrant (e.g., sodium starch glycolate, cross-linked carboxymethyl cellulose) surface-active or dispersing agent. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, or the like. Lubricants used in these dosage forms include sodium oleate, sodium stearate, magnesium stearate, sodium benzoate, sodium acetate, sodium chloride, or the like. Disintegrators include, for example, starch, methyl cellulose, agar, bentonite, xanthan gum, or the like. Molded tablets may be made by molding in a suitable machine a mixture of the powdered active ingredient moistened with an inert liquid diluent.

The tablets may optionally be coated or scored and may be formulated so as to provide slow- or controlled-release of the active ingredient. Tablets may also optionally be provided with an enteric coating to provide release in parts of the gut other than the stomach.

Formulations suitable for topical administration in the mouth wherein the active ingredient is dissolved or suspended in a suitable carrier include lozenges which may comprise the active ingredient in a flavored carrier, usually sucrose and acacia or tragacanth; gelatin, glycerin, or sucrose and acacia; and mouthwashes comprising the active ingredient in a suitable liquid carrier.

Topical applications for administration according to the method of the present embodiments include ointments, cream, suspensions, lotions, powder, solutions, pastes, gels, spray, aerosol or oil. Alternately, a formulation may comprise a transdermal patch or dressing such as a bandage impregnated with an active ingredient and optionally one or more carriers or diluents. To be administered in the form of a transdermal delivery system, the dosage administration will, of course, be continuous rather than intermittent throughout the dosage regimen.

The topical formulations may desirably include a compound that enhances absorption or penetration of the active ingredient through the skin or other affected areas. Examples of such dermal penetration enhancers include dimethylsulfoxide and related analogues.

The oil phase of an emulsion used to treat subjects in the present embodiments may be constituted from ingredients known to one of skill in the art in light of the present disclosure. An emulsion may comprise one or more emulsifiers. For example, an oily phase may comprise at least one emulsifier with a fat or an oil, with both a fat and an oil, or a hydrophilic emulsifier may be included together with a lipophilic emulsifier that acts as a stabilizer. Together, the emulsifier(s), with or without stabilizer(s), make up an emulsifying wax, and the wax together with the oil and/or fat make up the emulsifying ointment base that forms the oily dispersed phase of the cream formulations.

Emulsifiers and emulsion stabilizers suitable for use in the formulation include Tween 60, Span 80, cetosteryl alcohol, myristyl alcohol, glyceryl monostearate and sodium lauryl sulphate, paraffin, straight or branched chain, mono- or dibasic alkyl esters, mineral oil. The choice of suitable oils or fats for the formulation is based on achieving the desired cosmetic properties, the properties required and compatibility with the active ingredient.

Compounds of the present embodiments may also be administered vaginally, for example, as pessaries, tampons, creams, gels, pastes, foams or spray formulations containing appropriate carriers in addition to the active ingredient. Such carriers are known in the art in light of the present disclosure.

Formulations for rectal administration may be presented as a suppository with a suitable base comprising, for example, cocoa butter or a salicylate.

Formulations suitable for nasal administration may be administered in a liquid form, for example, nasal spray, nasal drops, or by aerosol administration by nebulizer, including aqueous or oily solutions of the active ingredient. Formulations for nasal administration, wherein the carrier is a solid, include a coarse powder having a particle size, for example, of less than about 100 microns, preferably less than about 50 microns, which is administered in the manner in which snuff is taken, i.e., by rapid inhalation through the nasal passage from a container of the powder held close up to the nose.

Formulations suitable for parenteral administration include aqueous and non-aqueous formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Extemporaneous injections solutions and suspensions may be prepared from sterile powders, granules and tablets of the kind previously described. Parenteral and intravenous forms may also include minerals and other materials to make them compatible with the type of injection or delivery system chosen.

In general, water, a suitable oil, saline, aqueous dextrose (glucose), or related sugar solutions and glycols such as propylene glycol or polyethylene glycols are suitable carriers for parenteral solutions. Solutions for parenteral administration preferably contain a water soluble salt of the active ingredient, suitable stabilizing agents and, if necessary, buffer substances. Antioxidizing agents, such as sodium bisulfite, sodium sulfite, or ascorbic acid, either alone or combined, are suitable stabilizing agents. Also used are citric acid salts thereof, or sodium EDTA. In addition, parenteral solutions may contain preservatives, such as benzalkonium chloride, methyl- or propyl-paraben, or chlorobutanol. Suitable pharmaceutical carriers are described in Remington, cited supra.

The combination or the fusion molecule may be micronized or powdered so that it is more easily dispersed and solubilized by the body. Processes for grinding or pulverizing drugs are well known in the art. For example, a hammer mill or similar milling device can be used.

Frequency of administration: The administration of fusion molecules or the combination of the present embodiments may be for a time period ranging between and including any of the following periods: one hour, one day, one week, one month, one year, and for life. The administration may occur once, twice, 3× or 4× per day. Generally, a fusion molecule or a combination of the present embodiments is administered on a daily basis one or more times a day, or one to four times a week, either in a single dose or separate doses during the day. Twice-weekly dosing over a period of several weeks is contemplated, and dosing may be continued over extended periods of time and possibly for the lifetime of the patient. However, the dosage and the dosage regimen will vary depending on the ability of the patient to sustain the desired and effective brain levels of the fusion molecule or of the combination of ampakine and nemdakine of the present embodiments.

Mode of Administration: A fusion molecule or a combination of an ampakine and a nemdakine of the present embodiments can be administered by a means that produces contact of the active agent with the agent's site of action in the brain, for example, suitable means including, but not limited to, oral, rectal, nasal, topical (including transdermal, aerosol, buccal or sublingual), vaginal, parenteral (including subcutaneous, intramuscular, intravenous or intradermal), intravesical, or injection via a catheter, shunt or a reservoir such as the Omaya reservoir. They can be administered by any conventional means available for use in conjunction with pharmaceuticals for the brain, either as individual but overlapping therapeutic agents or in a combination of therapeutics.

In each of the above-described methods, the administering may be in vivo, or may be ex vivo. In vivo treatment is useful for treating conditions in patients, and ex vivo treatment is useful for purging body fluids, such as blood, plasma, bone marrow, and the like, for return to the body.

Pharmaceutical Kits: Certain embodiments include pharmaceutical kits useful, for example, for memory enhancement. The kits comprise one or more containers containing a pharmaceutical composition comprising a therapeutically effective amount of a fusion molecule or a combination of a positive modulator of an NMDA receptor and a positive modulator of an AMPA receptor of the present embodiments. Such kits can further include, if desired, one or more of various conventional pharmaceutical kit components, such as, for example, containers with one or more pharmaceutically acceptable carriers, additional containers, etc., as will be readily apparent to those skilled in the art. Instruction, such as printed instructions for example, either as inserts or as labels, the instruction indicating quantities of the components to be administered, guidelines for administration, and/or guidelines for mixing the components, can also be included in the kit.

The following examples are presented to further illustrate various aspects of the present embodiments, and are not intended to limit the scope of the invention.

### EXAMPLE 1

### Synergistic Effect of a Combination of an Ampakine and a Nemdakine on STP and LTP Induction in Hippocampus

The present example demonstrates that two different combinations of an ampakine and a nemdakine at subthreshold concentrations produced an unexpected large facilitation of LTP under conditions where each individually had limited effect.

Adult mice hippocampal slices were prepared according to standard procedures as described in Dunwiddie and Lynch (J. Physiol. 276:353-367. 1978). Briefly, mice were decapitated following anesthesia, the brains rapidly removed and the hippocampus dissected. Transverse hippocampal slices (400 μm thick) were cut with a McIllwain tissue slicer (Stoelting Co., Wood Dale, Ill.). Slices were pre-incubated for at least 60 min in an artificial cerebrospinal fluid (aCSF) containing (in mM) NaCl (124), KCl (3), KH₂PO₄ (1.25), MgSO₄ (2.5), CaCl₂ (3.4), NaHCO₃ (26), glucose (10); and L-ascorbate (2) before being placed in a recording chamber. Slices were continuously perfused with aCSF containing no added drug, 20 μM CX546, 2 μM D, L-serine, or a combination of 20 μM CX546 and 2 μM D, L-serine. A stimulating electrode was placed in the stratum radiatum at the junction of CA3 and CA1 and a recording electrode was placed in the stratum radiatum of CA1. Baseline recording with a stimulation frequency of 0.033 Hz was done for at least 10 min. After obtaining a stable baseline for 10 min, LTP was elicited in CA1 stratum radiatum by stimulation of the Schaffer collateral pathways by using high frequency stimulation of 5 bursts of 3 pulses at 100 Hz, with the bursts delivered at the theta frequency, i.e., 5 Hz. Low frequency stimulation was resumed and responses recorded for at least 30 min.

Slopes of the extracellular postsynaptic potentials (EPSPs) were calculated and expressed as percent of the average values recorded during the 10 min baseline period. Results were averaged and expressed as means±s.e.m.

EPSPs recorded in control slices (perfused in the absence of added drugs) exhibited typical short-term potentiation (STP) and slowly decayed to stabilize after 30 min to reach levels about 20% above baseline values (FIG. 2). Perfusion of slices with 20 μM CX546 alone, or 2 μM D, L-serine alone, did not modify the levels of potentiation as compared to control slices. Therefore, results from control slices were combined with those from slices perfused with CX546 (20 μM) alone and D, L-serine (2 μM) alone. The control data therefore represent the means±s.e.m., of 15 slices and are provided in FIG. 2 (solid triangles).

In contrast, perfusion of slices with a combination of 20 μM CX546 and 2 μM D, L-serine resulted in a massive increase in STP and LTP (FIG. 2 (open circles)). The data represent the means±s.e.m., of 6 slices for the combination of 20 μM CX546 and 2 μM D, L-serine (results were statistically significant with ANOVA with repeated measures).

The data of FIG. 2 demonstrate an unexpected synergy in LTP provided by the combination of an ampakine and a nemdakine. The combination produced a large facilitation of LTP under conditions producing only limited LTP by each agent separately. D-serine is known to facilitate LTP at a concentration of 10 μM which is 10 times the concentrations used herein (2 μM of D, L-serine is used herein, thereby providing 1 μM D-serine), and CX546 is known to facilitate LTP at a concentration of 200 μM which is also 10 times the concentrations used herein (20 μM of CX546 is used herein). The results demonstrate that the combination provided an unexpected effect at 1/10^{th} the concentration of these compounds used separately.

A second study tested a combination of piracetam at 250 μM (a benzothiadiazine and an ampakine) and D, L-serine at 2 μM (a nemdakine). Hippocampal slices were prepared as cited above and electrophysiological recording in field CA1 demonstrated that the combination produced a significantly larger degree of LTP as compared with either piracetam alone, D, L-serine alone or control.

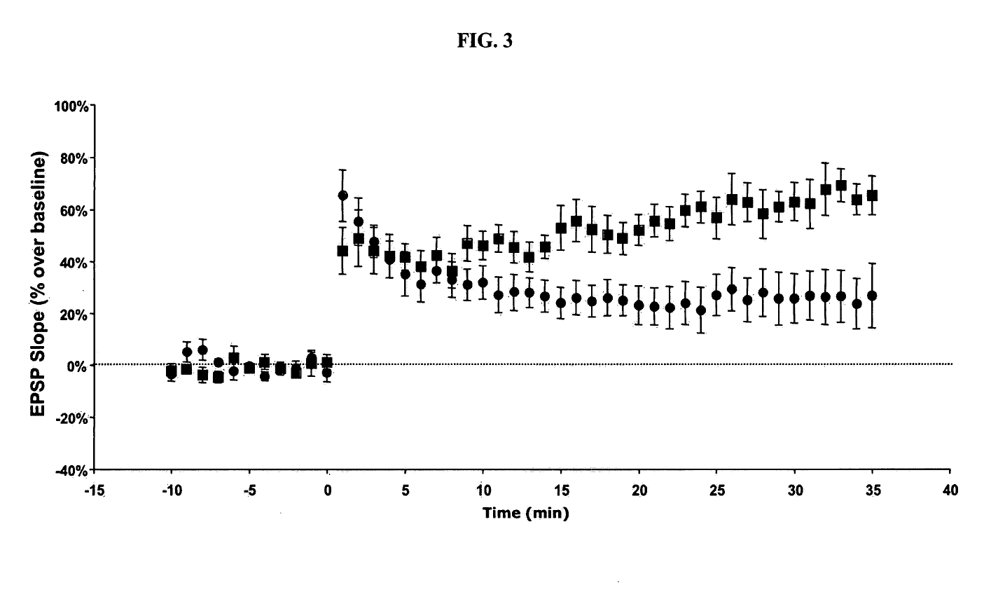

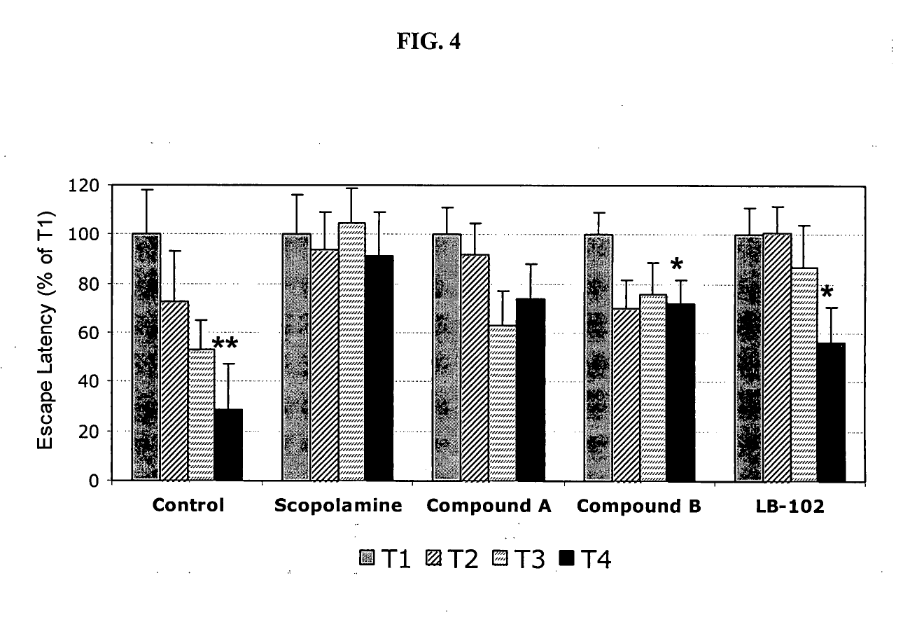

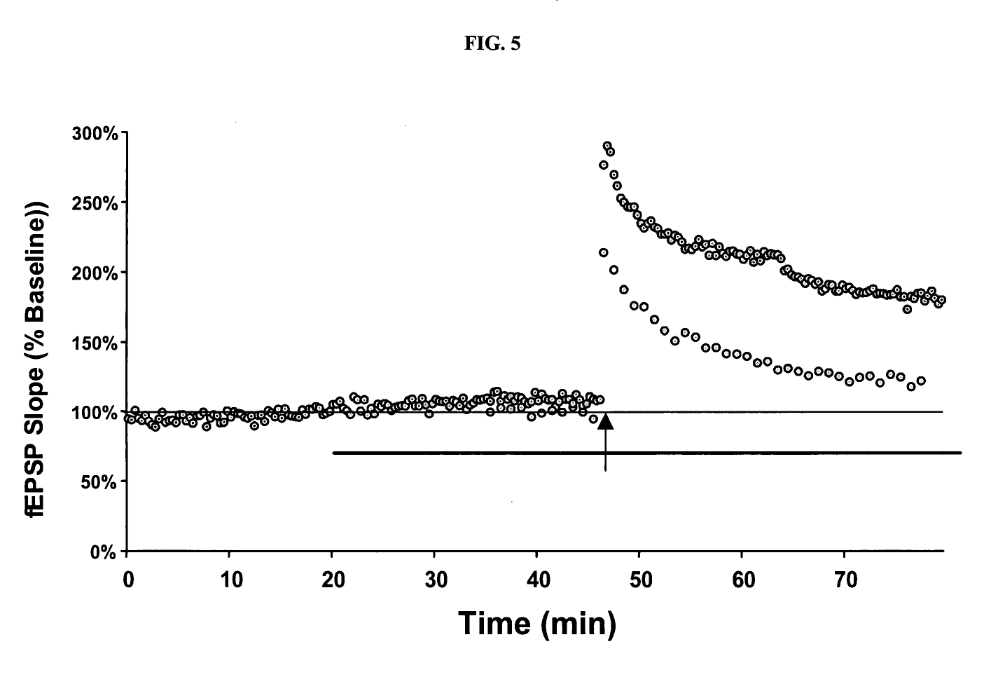

In particular, slices were prepared as cited above and EPSPs were evoked in CA1 stratum radiatum by stimulation of the Schaffer collateral pathways once every 30 seconds. At t=0, high frequency stimulation (5 bursts of 3 pulses at 100 Hz with bursts delivered at 5 Hz) was delivered, and low frequency stimulation resumed. Slopes of EPSPs were calculated and expressed as percent over the average values during the 10 minute baseline. Results from control slices were combined with results from slices perfused with piracetam (250 μM) alone and D, L-serine (2 μM) alone, since these drugs by themselves did not produce any significant effect on the responses. The data of FIG. 3 therefore represent the means±s.e.m., of 8 slices for control conditions (●), and 6 slices for the combination of 250 μM piracetam and 2 μM D, L-serine (▪). Results were statistically significant with ANOVA with repeated measures.

The data of FIG. 3 demonstrate an unexpected synergy in LTP provided by this second example of a combination of an ampakine and a nemdakine. The combination produced a large facilitation of LTP under conditions producing no significant effect by each agent separately.

### EXAMPLE 2

### Synergistic In Vivo Effect of a Combination of an Ampakine and a Nemdakine

The present example demonstrates that a combination of an ampakine, piracetam, and a nemdakine, D-serine, at subthreshold doses produced a significant reversal of learning and memory deficits in vivo under conditions where each individually has borderline effect at doses almost three times higher than the amount used in combination.

Learning and memory performance: Learning and memory performances were tested in the conventional Morris water maze according to the following procedure. The Morris maze includes a circular water tank (150 cm in diameter) filled with water and maintained at 27° C. with an escape platform (15 cm in diameter) 18 cm from the perimeter always in the same position 2 cm beneath the surface of the water. The water is made opaque by addition of milk powder rendering the platform invisible.

Male Wistar rats (200-230 g body weight, maintained in standard laboratory conditions) were given a single training session on one day. A training session consists of 4 consecutive trials (T1-T4) in the Morris water maze separated by 60 seconds. For each trial the animal is placed in the maze at one of two starting points equidistant from the escape platform and allowed to find the escape platform. The animal is left on the escape platform for 60 seconds before starting a new trial. If the animal does not find the platform within 120 seconds, the animal is removed from the water and placed on the platform for 60 seconds before beginning the next trial. During the 4 trials the animals start the maze twice from each starting point in a randomly determined order per animal. The time the animal takes to find the escape platform is referred to as the “escape latency.”

Measure of escape latency in control and test animals: The ampakine, the nemdakine, and the combination of ampakine and nemdakine of the present invention were administered p.o. to test animals 60 minutes before a test session in the maze (t=−60 min). Scopolamine was administered i.p. to induce learning and memory deficits 30 minutes later, i.e., 30 minutes before the test session in the maze (t=−30 min). Scopolamine induces amnesia as shown by the failure of scopolamine-treated animals to reduce their escape latencies from trial to trial.

The test drug treatment protocol was as follows:
control (physiological saline);
scopolamine alone (FIG. 4, 0.5 mg/kg i.p.) at t=−30 min;
piracetam alone (designated Compound A in FIG. 4, 500 mg/kg p.o.) at t=−60 min, followed by scopolamine (0.5 mg/kg i.p.) at t=−30 min;
D-serine alone (designated Compound B in FIG. 4, 1000 mg/kg p.o.) at t=−60 min, followed by scopolamine (0.5 mg/kg i.p.) at t=−30 min; and
a combination of piracetam, 150 mg/kg, and D-serine, 300 mg/kg, (p.o., the combination designated as LB-102 in FIG. 4) at t=−60 min, followed by scopolamine (0.5 mg/kg i.p.) at t=−30 min.

The results of the piracetam, D-serine and the combination, LB102, were compared with the scopolamine and the normal control group. There were 12 animals per group (N=12). Each animal was given 4 successive trials.

The principal measure taken at each trial was the escape latency. Measures consisted in the time the animals took to find the platform and were analyzed by ANOVA with repeated measures. To facilitate visual representation of the results, the data were also normalized by expressing them as percentage of the average values of the escape latency at the first trial (T1) in each group.

Results: As shown in FIG. 4, control rats learned the location of the platform as reflected by a decrease in escape latency across the four successive trials.

In contrast, scopolamine-treated rats exhibited a major impairment as they failed to improve their performance across the 4 trials (data labeled “Scopolamine” of FIG. 4).

Animals treated with piracetam followed by scopolamine exhibited a trend towards a reversal of scopolamine-induced learning impairment, although this effect did not reach statistical significance (data labeled “Compound A, ” FIG. 4).

Treatment with D-serine followed by scopolamine demonstrated a statistically significant reversal of scopolamine-induced impairment (data labeled “Compound B, ” FIG. 4).

Animals treated with the combination of piracetam and D-serine followed by scopolamine demonstrated a significant reversal of the effect of scopolamine and, at the last trial (T4), the combination treatment resulted in a 50% reversal of the effect of scopolamine (data labeled “LB-102, ” FIG. 4). This effect is demonstrated at doses of piracetam and D-serine in the combination LB-102 that are ⅓ the doses used in the treatment with each individual compound.

The results of this study provide a clear proof-of-principle as set forth herein. The in vivo results as presented here support the results provided by the in vitro studies of Example 1. The combination of an ampakine and a nemdakine provides a significant degree of synergy in reversal of scopolamine-induced learning and memory impairment since a dose of the combination has a concentration of each ingredient that is ⅓ the concentration that provides a minimal degree of facilitation when administered alone. The present protocol and test data primarily address a deficit in working memory.

### EXAMPLE 3

### A Fusion Molecule that Combines Functionalities of the Ampakine, CX546, and the Nemdakine, D-Serine

The present example provides a new series of molecules that combines the functionality of an ampakine with the functionality of a nemdakine. As an example, a synthesis scheme is provided for a molecule designated LB-217-1c, C-alpha-[N-(3, 4-dioxyethylenebenzoyl)-piperidine-4-yl]methyl-serine. The official name according to the IUPAC rules is (R)-2-amino-3-[1-(2, 3-dihydro-benzo[1, 4]dioxine-6-carbonyl)-piperidin-4-yl]-2-hydroxymethyl-propionic acid.

LB-217-1c is a fusion molecule of two molecules that, in combination, are shown by Example 1 to facilitate LTP formation. The principle of this fusion molecule is to link a molecule of CX546 to a molecule of D-serine by means of a one-carbon spacer. However, a number of attachment sites are possible on each molecule. According to a structural homology analysis performed on known ampakines and the knowledge of binding determinants as obtained from crystallographic studies on the NR1 subunit of the NMDA receptor, the possible sites of fusing the two molecules and minimizing the risk of losing bonds within their respective binding pockets are those labeled as Rα, Rβ, and Rγ in case of CX546 and R1-R4 in case of D-serine as shown below.

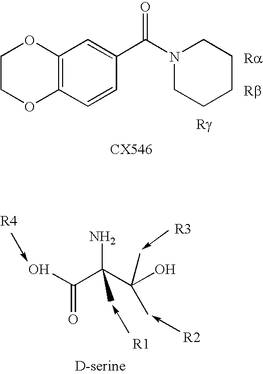

Thus, one part of the fusion molecule has the functionality of CX546 and the other part of the molecule has the functionality of D-serine. Both building blocks are linked to each other with a one-carbon spacer between R1 of CX546 and R1 of D-serine as follows.

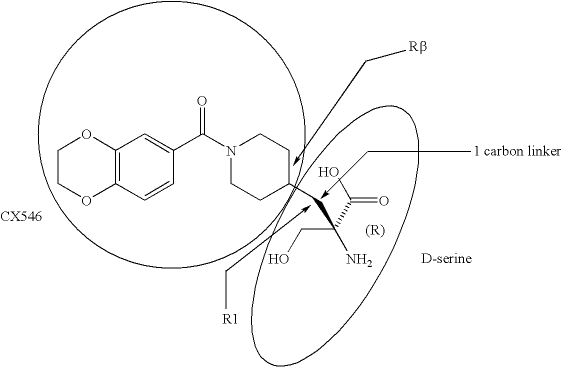

The synthesis scheme is as follows.

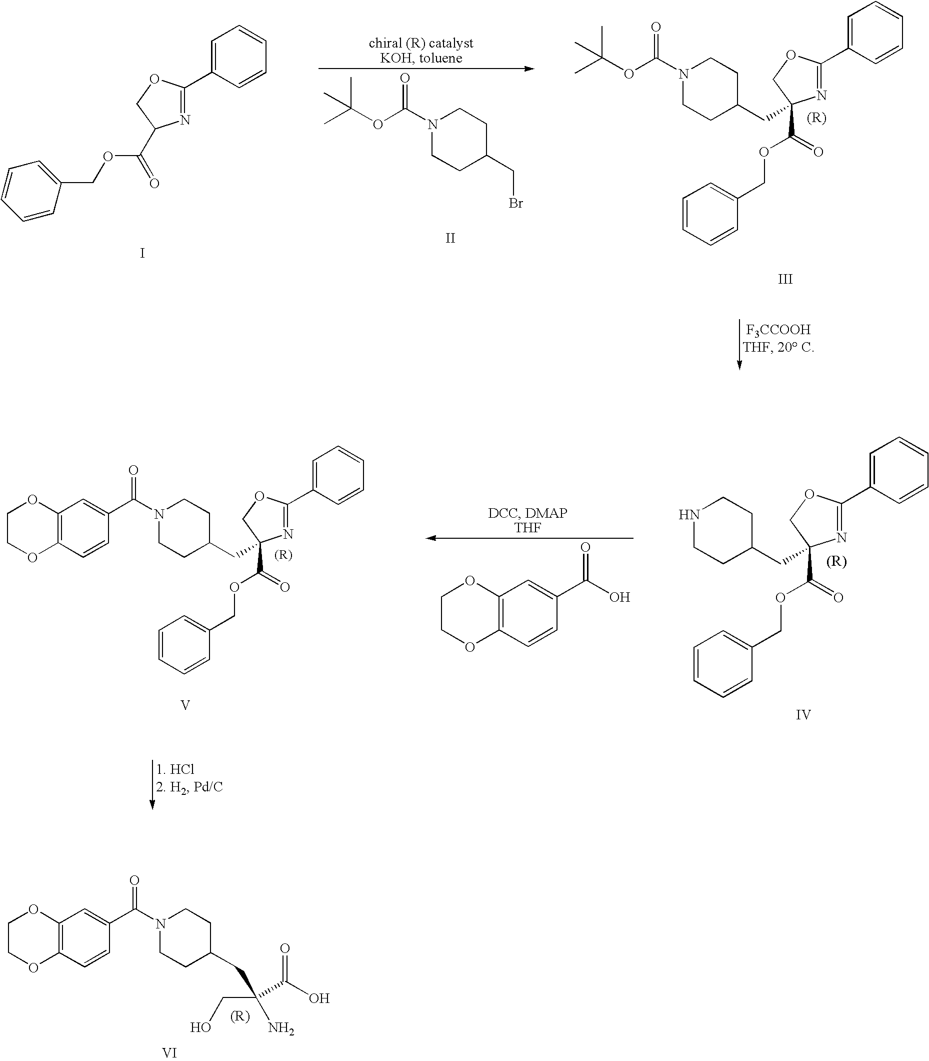

Reactant I is a phenyl oxazoline derivative of serine benzyl ester. A 4-bromomethyl piperidine t-butyl ester II is added in the presence of a chiral catalyst to form intermediate III. Chiral phase-transfer catalysts include hydrocinchonidine-derived catalysts including an (S)-binaphthol derivative available from Sigma-Aldrich (St. Louis, Mo.) as described by Jew, S., et al., (Angew. Chem. Int. Ed. 2004, 43:2382 and references cited therein). Since D-serine has R-chirality, the R-enantiomer of catalyst 4a of the Jew et al. reference is selected for the D-serine derivative. In one alternative, step one is carried out without a chiral catalyst. In that case, the resulting stereomeric mixture is separated on a chiral column. A second alternative is to use a chiral ester instead of a benzyl ester as reactant I which would lead to separable diastereomers. A third alternative is to use 3-bromomethyl piperidine t-butyl ester which is commercially available as a racemate and leads to separable diastereomer products. Cleavage of the t-butyloxycarbonyl group of III with trifluoroacetic acid leads to the N-unprotected piperidine derivative IV which is coupled with 2, 3-dihydro-benzo(1, 4)dioxine-6-carboxylic acid to form the amide V. Hydrolysis of V with hydrochloric acid followed by hydrogenation with hydrogen and palladium on charcoal as catalyst gives 2-amino-3-(1-(2, 3-dihydro-benzo(1, 4)dioxine-6-carbonyl)-piperidin-4-yl)-2-hydroxymethyl-propionic acid (VI). While the above scheme shows the bridging carbon as provided by a 4-bromomethyl piperidine t-butyl ester, one of ordinary skill in the art would recognize that two, three, four, or five bridging carbons would be provided by a 4-bromoalkyl piperidine t-butyl ester where the alkyl is a short chain alkyl such as an ethyl, propyl, butyl, or pentyl.

Molecule VI is LB-217-1c, C-alpha-[N-(3, 4-dioxyethylenebenzoyl)-piperidine-4-yl]methyl-serine.

Theoretical physico-chemical properties of LB-217-1c are as follows:
ClogP: −1.045
CMR: 9.321
Molecular Weight: 364.40
H-bond acceptors: 8
H-bond donors: 4
Amide groups: 1
log BB: −1.8
Pm: 52.48 [10-5 cm/min]
log Pm: −3.28
Absorption Fm: 98.18
PSA: 122.32
Molecular Weight=364.40
Exact Mass=364
Molecular Formula=C₁₈H₂₄N₂O₆
Molecular Composition=C, 59.33%; H, 6.64%; N, 7.69%; O, 26.34%

One of ordinary skill in the art of organic synthesis recognizes that an entire series of derivatives can be produced by routine modifications of the above cited scheme. Such derivatives are also provided by the present invention as molecules having the dual functionality described herein.

The theoretical docking of LB-217-1c within the cyclothiazide (CTZ) site of the AMPA GluR2 and the D-serine binding cleft of the NR1 subunit of the NMDA receptor was examined using VLifeMDS™ software (Vlife Sciences Technologies Pvt Ltd, Aundh, Pune, India). The 3-D structure of the GluR2 S1S2 segment was constructed from data deposited into the Protein Data Base (PDB code: 1LBC) (Sun et al., Nature 417:245-253, 2002). An apo structure was first constructed. Two CTZ molecules were then docked into this apo structure at the dimer interface. Similarly, two molecules of LB-217-1c were then docked into the same site of the apo structure of the GluR2 dimer construct. The positioning of LB-217-1c, description of bonds, and energy of stabilization were compared to those of CTZ. The results indicate that LB-217-1c is clearly predicted to bind within the CTZ site. Two molecules of LB-217-1c can take the position of CTZ and establish a number of hydrogen and hydrophobic bonds. Although there were fewer bonds predicted with theoretical docking of LB-217-1c than with CTZ, 83% of the residues that form bonds were in common with those of CTZ.

With regard to the D-serine portion of LB-217-1c, the 3-D structure of the D-serine binding pocket was constructed from the crystal structure of the D-serine binding pocket of the NR1 subunit (PDB code: 1PB8) (Furukawa et al., EMBO J. 22:2873, 2003). The positioning of LB-217-1c, description of bonds, energy of stabilization and closure distance of the binding cleft were compared to those of D-serine and the antagonist 5, 7-dichlorokynurenic acid (DCKA). The observations indicate that LB-217-1c is likely to bind within the D-serine binding cleft. The positioning and binding pattern suggest that LB-217-1c resembles more the agonist D-serine than the antagonist DCKA.

Further dual function molecules of the present invention are provided by the various positions of attachment for CX546: Rα, Rβ, and Rγ and the positions of attachment for D-serine: R1, R2, R3, and R4. The combination of attachments provides 12 molecules with the code number assigned as follows:

| | R1 | R2 | R3 | R4 |
|---|---|---|---|---|
| Rα | 213 | 214 | 215 | 216 |
| Rβ | 217 | 218 | 219 | 220 |
| Rγ | 221 | 222 | 223 | 224 |

Dual function molecule 217 was exemplified herein (LB217-1c).

By analogy, one can examine other positive modulators of AMPA receptors, such as piracetam cited herein, or a biarylpropylsulfonamide of Example 4 infra, for attachment sites to a positive modulator of an NMDA receptor to provide further series of fusion molecules having the dual functionality provided herein.

### EXAMPLE 4

### A Fusion Molecule that Combines Functionalities of the Ampakine, LY404187, and the Nemdakine, D-Serine

The present example provides for fusion of the functionality of an ampakine that is more potent than the ampakine of Example 3 with the functionality of a nemdakine into one molecule. A synthetic scheme is provided herein for a molecule designated LB-253-4c. The official name according to the IUPAC rules for LB-253-4c is 2-amino-2-hydroxymethyl-6-{4′-[1-methyl-2-(propane-2-sulfonylamino)-ethyl]-biphenyl-4-ylamino}-hexanoic acid.

The ampakine functionality of LB-253-4c is a biarylpropylsulfonamide (Ornstein et al., ibid). One of the best characterized biarylpropylsulfonamides, LY404187, has been shown to increase AMPA-induced currents in cerebellar Purkinje cells as well as in hippocampal pyramidal neurons with an EC₅₀ value of 30 nM-300 nM (Gates et al., ibid). LY404187 is therefore about 1000 times more potent than CX546 and, in addition, LY404187 has a mode of action that differs from that of cyclothiazide and possibly from that of other PARMs (Quirk, J. C. et al., J Neuroscience, 23(34):10953-10962, Nov. 26, 2003). The new fusion molecule, LB-253-4c, therefore, likely has a different mode of action compared to that of cyclothiazide, a mode of action more directed towards deactivation instead of desensitization.

From a study of the structure-activity relationship of the compounds of Ornstein (Ornstein, et al., 2000, ibid), a derivative of LY404187 designated 22h was chosen to provide the ampakine functionality of the new fusion molecule. The structural difference between LY404187 and the 22h derivative is that 22h has an amino substituent on the distal aromatic ring of the biphenyl group instead of a cyano substituent. The 22h derivative is about 2 fold more active than LY404187 (130 nM vs 290 nM) in the potentiation of the L-glutamate mediated currents on HEK cells expressing GluR4 flip (Ornstein, et al., 2000, ibid).

The new series of fusion molecules provided herein combine the 22h derivative of the biarylpropylsulfonamide series with D-serine. Based on the docking study of Example 3 with LB-217-1c, a spacer of four carbons is introduced between the D-serine and the amino group of 22h to allow full closure of the binding cleft of the D-serine binding pocket.

D-serine is attached to 22h via the Rα position of 22h. This attachment keeps the α-carboxyl and amino groups of D-serine free for receptor binding as well as the β-hydroxyl group as shown in the 3-D structure studies with Sun et al. (2002, ibid).

With regard to the 22h ampakine portion of the fusion molecule, the sulfonamide structure SO₂NH₂ is considered a determinant for the PARM activity, since this motif is a common feature of several very active PARMs. Further, structure-activity relationship studies demonstrated that the absence of the SO₂NH₂ motif in the biarylpropylsulfonamide series of molecules led to a dramatic loss of activity (Ornstein et al., 2000 ibid). Therefore D-serine is attached via a four-carbon spacer to the amino group of the most distal phenyl ring which leaves the sulphonamide moiety free for interactions. Similarly, the isopropyl substituent of the sulphonamide (—CH(CH₃)₂) appears to confer the highest potency for modulating AMPA-mediated responses. The data of Ornstein (Ornstein et al., 2000 ibid) regarding substitutions of the NH₂ group of 22h by groups other than halogenates suggest that such substitutions provide acceptable levels of potency. Therefore, D-serine was attached at position R1 to 22h in position Rα as depicted below.

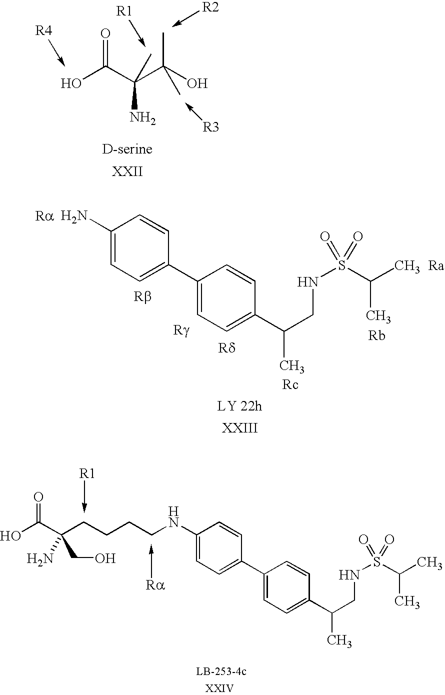

Note that 27 other possible combinations of D-serine and derivative 22h exist as follows:

| | R1 | R2 | R3 | R4 |
|---|---|---|---|---|
| Rα | 253 | 254 | 255 | 256 |
| Rβ | 257 | 258 | 259 | 260 |
| Rγ | 261 | 262 | 263 | 264 |
| Rδ | 265 | 266 | 267 | 268 |
| Ra | 269 | 270 | 271 | 272 |
| Rb | 273 | 274 | 275 | 276 |
| Rc | 277 | 278 | 279 | 280 |

Fusion molecule LB-253-4c is synthesized according to the following scheme which uses known methods (Jew et al., 2004, ibid; Ornstein et al., 2000, ibid) modified as set forth herein. Racemic compounds may be synthesized before the preparation of enantiomerically pure target molecules.

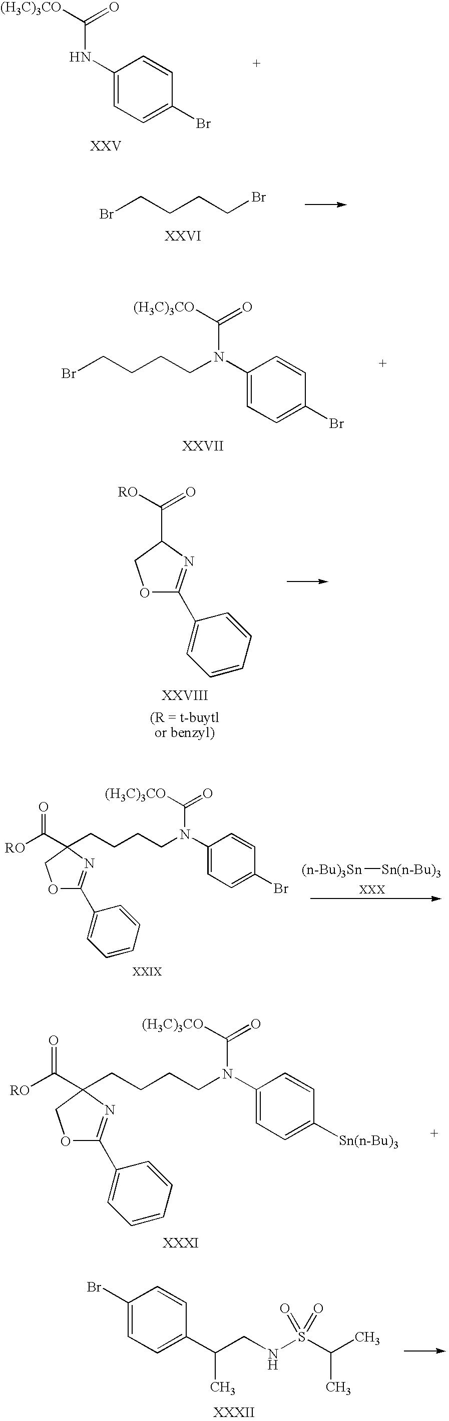

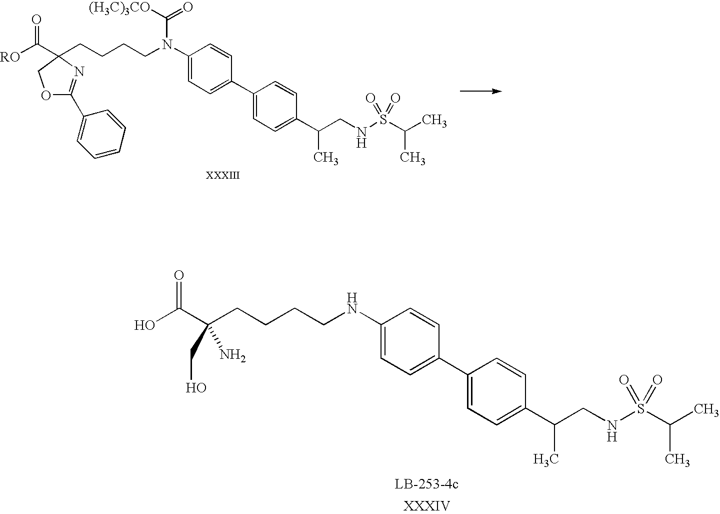

Alkylation of (4-bromo-phenyl)-carbamic acid tert-butyl ester (XXV) (precursor for (4-tributylstannanyl-phenyl)-carbamic acid tert-butyl ester of Ornstein et al., 2000, ibid) with 1, 4-dibromo-butane in the presence of a base leads to (4-bromo-butyl)-(4-bromo-phenyl)-carbamic acid tert-butyl ester (XXVII) which is used as alkylating agent for 2-phenyl-4, 5-dihydro-oxazole-4-carboxylic acid tert-butyl ester (or benzyl ester) (XXVIII) in the next step (analogous to the method of S. Jew et al., 2004, ibid) to provide 4-{4-[(4-bromo-phenyl)-tert-butoxycarbonyl-amino]-butyl}-2-phenyl-4, 5-dihydro-oxazole-4-carboxylic acid ethyl ester (XXIX). Conversion of XXIX to the stannane 4-{4-[tert-butoxycarbonyl-(4-tributylstannanyl-phenyl)-amino]-butyl}-2-phenyl-4, 5-dihydro-oxazole-4-carboxylic acid ethyl ester (XXXI) is achieved according to standard procedures (e.g. analogous to the conversion of (4-bromo-phenyl)-carbamic acid tert-butyl ester to (4-tributylstannanyl-phenyl)-carbamic acid tert-butyl ester of Ornstein et al.). Pd-mediated coupling of XXXI with propane-2-sulfonic acid (2-(4-bromo-phenyl)-propyl)-amide (XXXII; described in Ornstein et al.) gives 4-(4-(tert-butoxycarbonyl-{4′-(1-methyl-2-(propane-2-sulfonylamino)-ethyl)-biphenyl-4-yl}-amino)-butyl)-2-phenyl-4, 5-dihydro-oxazole-4-carboxylic acid ethyl ester (XXXIII) which is hydrolysed to the free amino acid 2-amino-2-hydroxymethyl-6-{4′-[1-methyl-2-(propane-2-sulfonylamino)-ethyl]-biphenyl-4-ylamino}-hexanoic acid (LB-253-4c; XXXIV).

### EXAMPLE 5

### LTP Induction in Hippocampus by a Fusion Molecule that Combines Functionalities of an Ampakine and a Nemdakine

The present example provides a new fusion molecule that combines the functionality of an ampakine with the functionality of a nemdakine together with a derivatization of one of the two functional components. In this example, the fusion molecule is a derivative of CX546, D-serine and an alkyl group with 1 carbon bridging both components. The compound shown below is designated LB-302 and has an IUPAC name of 2-amino-3-(1-benzyl-1, 2, 3, 6-tetrahydro-pyridin-4-yl)-2-hydroxymethyl-propionic acid. LB-302 is synthesized as follows.

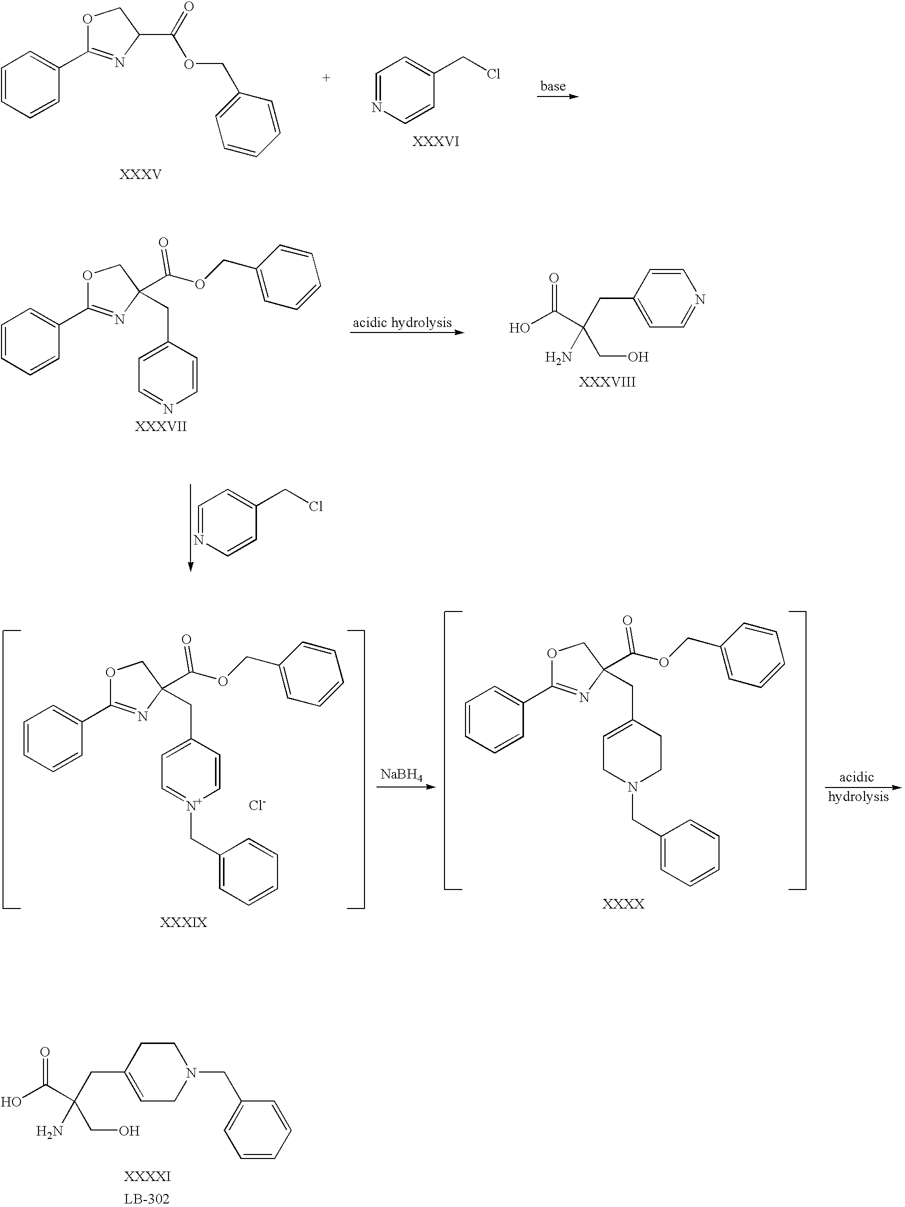

2-Phenyl-4, 5-dihydro-oxazole-4-carboxylic acid benzyl ester (XXXV) is alkylated with 4-chloromethyl-pyridine (XXXVI) under basic conditions (e.g. as reported for the reaction of the corresponding 2-phenyl-4, 5-dihydro-oxazole-4-carboxylic acid tert-butyl ester and chloromethyl-benzene, Jew et al., 2004, ibid) to yield 2-phenyl-4-pyridin-4-ylmethyl-4, 5-dihydro-oxazole-4-carboxylic acid benzyl ester (XXXVII) which is hydrolyzed with acid to the 2-amino-2-hydroxymethyl-3-pyridin-4-yl-propionic acid (XXXVIII).

Quarternization of (XXXVII) with chloromethyl-pyridine (XXXVI) leads to 1-benzyl-4-(4-benzyloxycarbonyl-2-phenyl-4, 5-dihydro-oxazol-4-ylmethyl)-pyridinium chloride (XXXIX) which is not isolated in pure form but reduced directly with sodiumborohydride to provide 4-(1-benzyl-1, 2, 3, 6-tetrahydro-pyridin-4-ylmethyl)-2-phenyl-4, 5-dihydro-oxazole-4-carboxylic acid benzylester (XXXX). Acid hydrolysis of (XXXX) gives 2-amino-3-(1-benzyl-1, 2, 3, 6-tetrahydro-pyridin-4-yl)-2-hydroxymethyl-propionic acid (XXXXI) which is LB-302.

LB-302 has been tested for formation of LTP as measured on mice hippocampal slices according to the procedure described in Example 1.

Slices were incubated in control medium. LB-302 (100 μM) was added to the medium at t=20 min and LTP was induced as in Example 1 by using high frequency stimulation of 5 bursts of 3 pulses at 100 Hz, with the bursts delivered at the theta frequency, i.e., 5 Hz delivered at t=47 min. As shown in FIG. 5, LB-302 produced a small increase in baseline response, which was expected if the molecule is a positive AMPA receptor modulator, and a dramatic increase in LTP amplitude (FIG. 5). Fusion molecule LB-302 displays a pattern of response that is similar to the the combination of CX546 plus D, L-serine of Example 1 and FIG. 2.

Other embodiments of the present invention will be apparent to those skilled in the art from a consideration of this specification or practice of the embodiments disclosed herein. However, the foregoing specification is considered merely exemplary of the present invention with the true scope and spirit of the invention being indicated by the following claims.

The references cited herein, to the extent that they provide exemplary procedural or other details supplementary to those set forth herein, are specifically incorporated by reference.

As used herein and unless otherwise indicated, the terms “a” and “an” are taken to mean “one”, “at least one” or “one or more”.

What is claimed is:

1. A method for enhancing memory of a subject, the method comprising:
administering to the subject a therapeutically effective amount of a combination of a positive modulator of AMPA receptors and a positive modulator of NMDA receptors,
wherein each modulator of the combination is present at a subtherapeutic dose for effecting memory enhancement.

2. The method of claim 1 wherein the subject has symptoms of a neurodegenerative disease.

3. The method of claim 1 wherein the subject has symptoms of cognitive impairment due to aging, Alzheimer's disease, dementia, schizophrenia, attention deficit hyperactivity disorder, or Parkinson's disease.

4. The method of claim 1 wherein the subject is in need of improvement in performance of a cognitive task.

5. The method of claim 1 wherein the method of administering is oral, nasal, topical, via injection, or via a catheter.

6. The method of claim 1 wherein the positive modulator of an AMPA receptor comprises an azepine, a benzamide, benzoylpiperidine, benzoylpyrrolidine, benzoxazine, benzothiadiazide, benzothiadiazine, biarylpropylsulfonamide, pyrrolidinone, pyrroline, tetrahydropyridine, phenoxyacetamide, sulfur-containing organic nitrate ester, lectin, a salt thereof, an ester thereof, a precursor thereof, a metabolite thereof, a derivative thereof, a racemic mixture thereof, or a combination thereof.

7. The method of claim 1 wherein the positive modulator of an NMDA receptor is an L-alanine, D-alanine, D-cycloserine, N-methylglycine, L-serine, D-serine, N, N, N-trimethylglycine, 3-amino-1-hydroxypyrrolid-2-one (HA966), (R)-(N-[3-(4′-fluorophenyl)-3-(4′-phenylphenoxy)propyl])sarcosine (ALX5407), N-methyl-N-[3-[(4-trifluoromethyl)phenoxy]-3-phenyl-propyl]glycine (ORG 24598), polyamine, neurosteroid, a salt thereof, an ester thereof, a precursor thereof, a metabolite thereof, a derivative thereof, a racemic mixture thereof, or a combination thereof.

8. A composition comprising:
a combination of a positive modulator of AMPA receptors and a positive modulator of NMDA receptors in a therapeutically effective amount for effecting memory enhancement, and
a pharmaceutically acceptable carrier,
wherein each modulator of the combination is present at a subtherapeutic dose for effecting memory enhancement.

9. The composition of claim 8 wherein the positive modulator of an AMPA receptor comprises an azepine, a benzamide, benzoylpiperidine, benzoylpyrrolidine, benzoxazine, benzothiadiazide, benzothiadiazine, biarylpropylsulfonamide, pyrrolidinone, pyrroline, tetrahydropyridine, phenoxyacetamide, sulfur-containing organic nitrate ester, lectin, a salt thereof, an ester thereof, a precursor thereof, a metabolite thereof, a derivative thereof, a racemic mixture thereof, or a combination thereof.

10. The composition of claim 9 wherein the positive modulator of an AMPA receptor comprises a benzoylpiperidine and the benzoylpiperidine comprises a 1-(quinoxalin-6-ylcarbonyl)piperidine (CX516), 1-(1, 4-benzodioxan-6-ylcarbonyl)piperidine (CX546), 1-(4′-methoxymethylbenzoyl)piperidine, 1-(3′-methoxymethylbenzoyl)piperidine, 1-(4′-ethoxymethylbenzoyl)piperidine, 1-(4′-hydroxymethylbenzoyl)piperidine, 1-(4′-(3″, 4″-methylenedioxyphenoxy)-methylbenzoyl)piperidine, 1-(1, 3-benzodioxol-5-ylcarbonyl)-piperidine (1-BCP), (R, S)-1-(2-methyl-1, 3-benzodioxol-5-ylcarbonyl)-piperidine, 1-(1, 3-benzoxazol-6-ylcarbonyl)-piperidine, 1-(1, 3-benzimidazol-5-ylcarbonyl)-piperidine, a salt thereof, an ester thereof, a precursor thereof, a metabolite thereof, a derivative thereof, a racemic mixture thereof, or a combination thereof.

11. The composition of claim 9 wherein the positive modulator of an AMPA receptor comprises a benzoxazine, and the benzoxazine is 2H, 3H, 6aH-pyrrolidino[2″, 1″-3′2′]1, 3-oxazino[6′, 5′-5, 4]benzo[e]1, 4-dioxan-10-one (CX614), (R, S)-6-methoxymethyl-2, 3-dihydro-1H-pyrrolo[2, 1-b][1, 3]benzoxazine-9(3aH)-one, R, S)-7-methoxymethyl-2, 3-dihydro-1H-pyrrolo[2, 1-b][1, 3]benzoxazine-9(3aH)-one, 7, 8-dihydro-5aH, 10H-1, 3-dioxolo[4, 5-g]oxazolo[2, 3-b][1, 3]benzoxazin-10-one (1), 8, 9-dihydro-6aH, 11H-1, 4-dioxan[2, 3-g]oxazolo[2, 3-b][1, 3]benzoxazin-11-one, 7, 8-dihydro-2, 2-dimethyl-5aH, 10H-1, 3-dioxolo[4, 5-g]oxazolo[2, 3-b][1, 3]benzoxazin-10-one, 7, 8-dihydro-5aH, 10H-1, 3-dioxolo[4, 5-g]thiazolo[2, 3-b][1, 3]benzoxazin-10-one, 7, 8-dihydro-7-methyl-5aH, 10H-1, 3-dioxolo[4, 5-g]oxazolo[2, 3-b][1, 3]benzoxazin-10-one, 7, 8-dihydro-8-methyl-5aH, 10H-1, 3-dioxolo[4, 5-g]oxazolo[2, 3-b][1, 3]benzoxazin-10-one, 8, 9-dihydro-5aH, 7H, 10H-1, 3-dioxolo[4, 5-g][1, 3]oxazino[2, 3-b][1, 3]benzoxazin-11-one, 7, 8-dihydro-5a-methyl-10H-1, 3-dioxolo[4, 5-g]oxazolo[2, 3-b][1, 3]benzoxazin-10-one, a salt thereof, an ester thereof, a precursor thereof, a metabolite thereof, a derivative thereof, a racemic mixture thereof, or a combination thereof.

12. The composition of claim 9 wherein the positive modulator of an AMPA receptor comprises a benzothiadiazide and the benzothiadiazide is cyclothiazide, diazoxide, IDRA21, bendroflumethiazide, benzthiazide, buthiazide, chlorothiazine, epithiazide, hydrochlorothiazide, hydroflumethiazide, methylclothiazide, methalthiazide, polythiazide, trichlormethiazide, 5-ethyl-benzothiadiazide (compound D1), a salt thereof, an ester thereof, a precursor thereof, a metabolite thereof, a derivative thereof, a racemic mixture thereof, or a combination thereof.

13. The composition of claim 9 wherein the positive modulator of an AMPA receptor comprises a benzothiadiazine and the benzothiadiazine is 7-chloro-3-methyl-3-4-dihydro-2H-1, 2, 4 benzothiadiazine S, S, dioxide, (S)-2, 3-dihydro-[3, 4]cyclopentano-1, 2, 4-benzothiadiazine-1, 1-dioxide (S18986-1), a salt thereof, an ester thereof, a precursor thereof, a metabolite thereof, a derivative thereof, a racemic mixture thereof, or a combination thereof.

14. The composition of claim 9 wherein the positive modulator of an AMPA receptor comprises a benzoylpyrrolidine and the benzoylpyrrolidine comprises 2H, 5aH-pyrrolidino[2″, 1″-3′, 2′]1, 3-oxazino[6′, 5′-5, 4]benzo[d]1, 3-dioxolan-9-one (BDP-20, CX554), 1(1, 3-benzodioxol-5-ylcarbonyl)-pyrrolidine), a salt thereof, an ester thereof, a precursor thereof, a metabolite thereof, a derivative thereof, a racemic mixture thereof, or a combination thereof.

15. The composition of claim 9 wherein the positive modulator of an AMPA receptor comprises a biarylpropylsulfonamide and the biarylpropylsulfonamide comprises N-2-(4-(3-thienyl)phenyl)propyl-2-propanesulfonamide (LY392098), N-2-(4-(cyanophenyl)phenyl)propyl-2-propanesulfonamide (LY404187), (R)-4′-[1-fluoro-1-methyl-2-(propane-2-sulfonylamino)-ethyl]-biphenyl-4-carboxylic acid methylamide (LY503430), a salt thereof, an ester thereof, a precursor thereof, a metabolite thereof, a derivative thereof, a racemic mixture thereof, or a combination thereof.

16. The composition of claim 9 wherein the positive modulator of an AMPA receptor comprises a pyrrolidinone, and the pyrrolidinone comprises aniracetam, piracetam, oxiracetam, (R)-1-p-anisoyl-3-hydroxy-2-pyrrolidinone (AHP), a salt thereof, an ester thereof, a precursor thereof, a metabolite thereof, a derivative thereof, a racemic mixture thereof, or a combination thereof.

17. The composition of claim 9 wherein the positive modulator of an AMPA receptor comprises a tetrahydropyridine, and the tetrahydropyridine is 1-(1, 4-benzodioxan-5-ylcarbonyl)-1, 2, 3, 6-tetrahydropyridine, N-(4-dimethylamino)benzoyl-1, 2, 3, 6-tetrahydropyridine, 1-(1, 3-benzodioxol-5-ylcarbonyl)-1, 2, 3, 6-tetrahydropyridine, 1-(1, 3-benzoxazol-6-ylcarbonyl)-1, 2, 3, 6-tetrahydopyridine, 1-(1, 3-benzoxazol-5-ylcarbonyl)-1, 2, 3, 6-tetrahydropyridine, 1-(guinoxalin-6-ylcarbonyl)-1, 2, 3, 6-tetrahydropyridine, a salt thereof, an ester thereof, a precursor thereof, a metabolite thereof, a derivative thereof, a racemic mixture thereof, or a combination thereof.

18. The composition of claim 9 wherein the positive modulator of an AMPA receptor comprises a pyrroline, and the pyrroline comprises 1-(1, 4-benzodioxan-5-ylcarbonyl)-3-pyrroline, a salt thereof, an ester thereof, a precursor thereof, a metabolite thereof, a derivative thereof, a racemic mixture thereof, or a combination thereof.

19. The composition of claim 9 wherein the positive modulator of an AMPA receptor comprises a lectin, and the lectin comprises concanavalin A, wheat germ agglutinin, a salt thereof, an ester thereof, a precursor thereof, a metabolite thereof, a derivative thereof, a racemic mixture thereof, or a combination thereof.

20. The composition of claim 9 wherein the positive modulator of an AMPA receptor comprises a phenoxyacetamide, and the phenoxyacetamide comprises 4-[2-(phenylsulfonylamino)ethylthio]-2, 6-difluoro-phenoxyacetamide (PEPA), a salt thereof, an ester thereof, a precursor thereof, a metabolite thereof, a derivative thereof, a racemic mixture thereof, or a combination thereof.

21. The composition of claim 9 wherein the positive modulator of an AMPA receptor comprises a sulfur-containing organic nitrate ester, and the sulfur-containing organic nitrate ester comprises GT-21-005, a salt thereof, an ester thereof, a precursor thereof, a metabolite thereof, a derivative thereof, a racemic mixture thereof, or a combination thereof.

22. The composition of claim 9 wherein the positive modulator of an AMPA receptor comprises an azepine and the azepine comprises an (R)-7-fluoro-2, 3, 11, 11a-tetrahydro-1H, 5H-pyrrolo[2, 1-c][1, 4]benzoxazepine-5-one, (S)-7-fluoro-2, 3, 11, 11a-tetrahydro-1H, 5H-pyrrolo[2, 1-c][1, 4]benzox-azepine-5-one, (S)-9-fluoro-2, 3, 11, 11a-tetrahydro-1H, 5H-pyrrolo[2, 1-c][1, 4]-benzoxazepine-5-one, (R)-9-fluoro-2, 3, 11, 11a-tetrahydro-1H, 5H-pyrrolo[2, 1-c][1, 4]benzoxazepine-5-one, and (S)-6-fluoro-2, 3, 11, 11a-tetrahydro-1H, 5H-pyrrolo[2, 1-c][1, 4]benzoxazepine-5one, a salt thereof, an ester thereof, a precursor thereof, a metabolite thereof, a derivative thereof, a racemic mixture thereof, or a combination thereof.

23. The composition of claim 8 wherein the positive modulator of an NMDA receptor is an L-alanine, D-alanine, D-cycloserine, N-methylglycine, L-serine, D-serine, N, N, N-trimethylglycine, 3-amino-1-hydroxypyrrolid-2-one (HA966), (R)-(N-[3-(4′-fluorophenyl)-3-(4′-phenylphenoxy)propyl])sarcosine (ALX5407), N-methyl-N-[3-[(4-trifluoromethyl)phenoxy]-3-phenyl-propyl]glycine (ORG 24598), polyamine, neurosteroid, a salt thereof, an ester thereof, a precursor thereof, a metabolite thereof, a derivative thereof, a racemic mixture thereof, or a combination thereof.

24. A kit comprising a container containing the composition of claim 8 and instructions for using the composition for enhancing memory in a subject.

25. A composition comprising:
a fusion molecule having positive modulating activity for both AMPA receptors and NMDA receptors, the fusion molecule comprising an ampakine functional moiety fused to a nemdakine functional moiety; and
a pharmaceutically acceptable carrier.

26. The composition of claim 25 wherein the ampakine functional moiety is fused to a nemdakine functional moiety via a linking group.

27. The composition of claim 26 wherein the linking group comprises an alkyl group of 1, 2, 3, 4, or 5 carbons.

28. The composition of claim 25 wherein the ampakine functional moiety is derived from an azepine, a benzamide, benzoylpiperidine, benzoylpyrrolidine, benzoxazine, benzothiadiazide, benzothiadiazine, biarylpropylsulfonamide, pyrrolidinone, pyrroline, tetrahydropyridine, phenoxyacetamide, sulfur-containing organic nitrate ester, or a lectin.

29. The composition of claim 25 wherein the nemdakine functional moiety is derived from L-alanine, D-alanine, D-cycloserine, N-methylglycine, L-serine, D-serine, N, N, N-trimethylglycine, 3-amino-1-hydroxypyrrolid-2-one (HA966), (R)-(N-[3-(4′-fluorophenyl)-3-(4′-phenylphenoxy)propyl])sarcosine (ALX5407), N-methyl-N-[3-[(4-trifluoromethyl)phenoxy]-3-phenyl-propyl]glycine (ORG 24598), a polyamine, or a neurosteroid.

30. The composition of claim 28 wherein the ampakine functional moiety is derived from the benzoylpiperidine, CX546.

31. The composition of claim 28 wherein the ampakine functional moiety is derived from the biarylpropylsulfonamide derivative, LY404187-NH₂.

32. A fusion molecule having positive modulating activity for both AMPA receptors and NMDA receptors, the fusion molecule comprising an ampakine functional moiety fused to a nemdakine functional moiety.

33. The fusion molecule of claim 32 wherein the fusion molecule is LB-217-1c, (R)-2-amino-3-[1-(2, 3-dihydro-benzo[1, 4]dioxine-6-carbonyl)-piperidin-4-yl]-2-hydroxymethyl-propionic acid.

34. The fusion molecule of claim 32 wherein the fusion molecule is LB-253-4c, 2-amino-2-hydroxymethyl-6-{4′-[1-methyl-2-(propane-2-sulfonylamino)-ethyl]-biphenyl-4-ylamino}-hexanoic acid.

35. The fusion molecule of claim 32 wherein the fusion molecule is LB-302, 2-amino-3-(1-benzyl-1, 2, 3, 6-tetrahydro-pyridin-4-yl)-2-hydroxymethyl-propionic acid.

36. The fusion molecule of claim 32, having a structure A-1 or A-2: 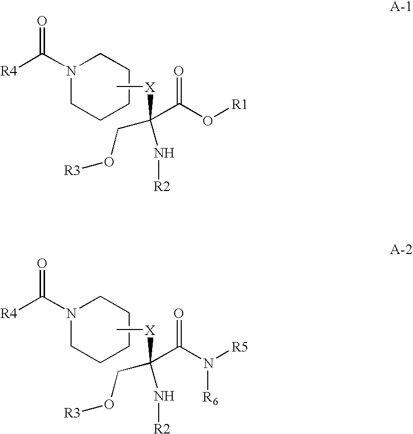
wherein
the piperidine ring is independently substituted in the 2-, 3- or 4-position;
X is an alkyl group of one to five carbon atoms;
R1 is C1-C4 alkyl, or aryl;
R2 and R3 are independently H, formyl or acyl;
R4 is a derivative of benzoic acid or of a five- or six-membered heterocyclic carboxylic acid with one or two rings; and
R5 and R6 are independently H, acyl, formyl, alkyl, or aryl.

37. The fusion molecule of claim 36 wherein the R4-C═O portion of the molecule is a thiophene-2-carboxylic acid, thiophene-3-carboxylic acid, pyridine-2-carboxylic acid, pyridine-3-carboxylic acid, pyridine-4-carboxylic acid, pyrimidine-2-carboxylic acid, pyrimidine-4-carboxylic acid, pyrimidine-5-carboxylic acid, pyrazine-2-carboxylic acid, 2, 3-dihydro-benzo(1, 4)dioxine-6-carboxylic acid, 1-quinoxalin-2-carboxylic acid, or 1-quinoxalin-6-carboxylic acid.

38. The fusion molecule of claim 37 wherein the R4-C═O portion of the molecule is substituted with one or two groups designated R7, and wherein each R7 is independently halo, alkoxy, alkyl, or cyano,
with the provisos that
R7 is other than methoxy or ethoxy when R4-C═O is a thiophene carboxylic acid, and
a halo group is in other than an ortho position relative to a nitrogen atom in a nitrogen-containing heterocycle.

39. The fusion molecule of claim 36 having structures VII or VIII as follows

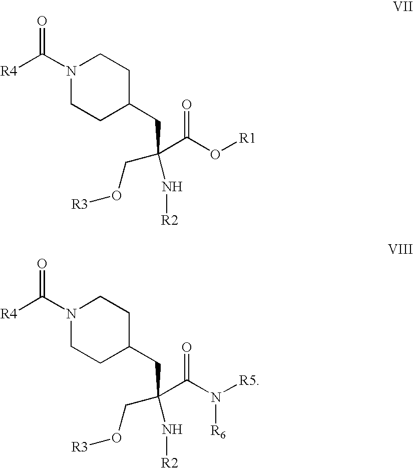

40. A method for enhancing memory of a subject, the method comprising administering to the subject a therapeutically effective amount of the composition of claim 25.
